(19) United States
(12) Patent Application Publication
(10) Pub. No.: US 2026/0242715 A1
(43) Pub. Date: Aug. 20, 2026

(54) HIGH VIABLE CELL DENSITY TRANSFECTION

(71) Applicant: Repligen Corporation, Waltham, MA (US)

(72) Inventors: René Gantier, Arlington, MA (US); Keen Chung, Northborough, MA (US); Karol Lacki, Höllviken (SE)

(21) Appl. No.: 19/161,910

(22) PCT Filed: Mar. 13, 2024
(86) PCT No.: PCT/US2024/019722

§ 371 Date: Sep. 3, 2025

Related U.S. Application Data
(60) Provisional application No. 63/452,067, filed on Mar. 14, 2023.

(51) Int. Cl. C12M 1/26; C12M 1/00; C12N 15/86

(52) U.S. Cl. CPC C12M 33/14; C12M 23/28; C12M 29/00; C12N 15/86; C12N 2750/14143; C12N 2750/14151

(57) ABSTRACT

Viral vectors for gene therapy can be produced using transfection in cell culture systems. The present disclosures describe cell culture systems that include cell retention devices and other components so that transfection occurs at high values of viable cell density (VCD). The present disclosures also include methods of operating cell culture systems at high values of viable cell density for optimized production of viral vectors.

### CROSS-REFERENCE TO RELATED APPLICATIONS

This application claims priority to and benefit of U.S. Provisional Patent No. 63/452,067, filed Mar. 14, 2023, the disclosure of which is incorporated by reference herein in its entirety.

### FIELD

The present disclosure relates generally to the production of viral vectors for gene therapy using transfection in cell culture systems.

### BACKGROUND

Transfection (TFN) is the process of inserting nucleic acids into eukaryotic cells, and has many applications in biotechnology. A typical process to produce viral vectors for gene therapy (see FIG. 1) starts with a cell culture conducted in a bioreactor until the viable cell density (VCD) reaches a set value for transfection. The production of virus particles by transfection can be done in transient expression mode. This step is usually performed using batch cell culture at low VCD (<5×10⁶ cells/mL) when cell growth is still in the logarithmic growth phase (see FIG. 2). DNA is mixed with transfection reagent at an optimized value.

After transfection, the cells start producing the virus either intracellularly (i.e., virus that is produced stay in the cells) or extracellularly (i.e., virus is excreted from the cells into the cell culture medium). At the end of the virus production phase, if the virus is present in the media, a cell clarification step is conducted, usually using filtration, to collect the virus. If the virus is present within the cells, a cell lysis step is needed before clarification to release the virus. The virus is then purified and formulated.

Various transfection methods are available, including physical, chemical, and viral methods. Chemical transfection methods are the commonly most used. In a typical chemical transfection method, charged polymer reagents that are able to penetrate cell membranes are mixed with nucleic acids to form DNA/reagent complexes. These complexes are then added to a cell culture to perform the transfection steps, during which the DNA/reagent complexes enter the cells.

High efficiency chemical transfection depends on a few critical parameters: (1) the ratio between nucleic acid and transfection reagent (i.e., mass of DNA per volume of transfection reagent); (2) the ratio between DNA and cells (i.e., mass of DNA per 1×10⁶ cells); and (3) the concentration of transfection reagent during complexation with DNA (i.e., volume of transfection reagent per complexation volume).

In order to conduct transfection at high VCDs in bioreactor vessels, a compromise must be made to use sub-optimal transfection conditions, e.g., by reducing the total volume of transfection mix by increasing the concentration of the transfection reagent to free up volume available for the cell culture. This will lead to (i) low transfection efficiency due to the cytotoxicity of transfection reagents, and (ii) the bioreactor not being used at full capacity.

Perfusion cell culture is a method where a cell retention device is used along with continuous exchange of media, resulting in higher VCDs and cell viabilities over much longer durations than what is achieved in fed-batch cell culture. In perfusion cell culture, cells can stay in logarithmic phase growth at higher cell density, so that transfection can be performed at higher cell density, which means more cells are present and more DNA is required. However, methods and systems to achieve effective transfection in a perfusion cell culture mode at high VCDs are limited.

### SUMMARY

The present embodiments include systems and methods to conduct transfection at high VCDs without compromising cell quality, transfection efficiency, or bioreactor capacity. To achieve effective transfection at high VCDs, the present embodiments include a single-use system and assembly including a microfiltration cell retention device and/or an ultrafiltration device operated in tangential flow filtration (TFF) or alternating tangential flow (ATF) modes. The present embodiments also include methods for using these systems to produce viral vectors for gene therapy.

Different configurations of the embodiments are described herein, along with different modes of operation to practice the embodiments. Examples of modes of operation include: cell culture volume reduction before transfection: continuous addition of transfection mix at constant bioreactor volume: continuous harvesting of the virus produced extracellularly during the production phase; and/or an integrated clarification step for processes where cells need to be lysed to release the virus produced intracellularly.

In one aspect, the present embodiments are directed to a method of producing viral vectors via cell transfection with reduction of cell culture volume, including: (a) providing a cell culture system, including: a continuous flow bioreactor; a cell retention device comprising a filtrate port that is fluidly connected to the continuous flow bioreactor; and a spent media collection vessel fluidly connected to the cell retention device; (b) performing cell culture of eukaryotic cells; (c) circulating the cells into the cell retention device and returning the cells into the continuous flow bioreactor until a viable cell density (VCD) in the continuous flow bioreactor reaches a target value suitable for transfection; (d) concentrating the cells, including: starting operation of cell retention device to circulate the cells into the cell retention device and returning the cells into the continuous flow bioreactor while continuously collecting the spent media from the permeate of the cell retention device until the volume in the continuous flow bioreactor is reduced by an amount equal to a volume of transfection reagent mixture to be added; (e) performing transfection of the cells, including the steps of: (i) feeding the volume of transfection reagent mixture into the continuous flow bioreactor while the spent media is not collected from the permeate of the cell retention device; and (ii) circulating the cells mixed with the transfection reagent mixture into the cell retention device and returning the cells into the continuous flow bioreactor for a period of time; and (f) producing virus in the continuous flow bioreactor by operating the bioreactor under virus production conditions; and (g) if perfusion after transfection is needed, continuously feeding a fresh supply of growth media through the continuous flow bioreactor while the spent media is continuously collected from the permeate of the cell retention device.

In some embodiments, the cell retention device is a microfiltration cell retention device. In some embodiments, the cell retention device is an ultrafiltration cell retention device. In some embodiments, the cell retention device is operated in tangential flow filtration (TFF) mode. In some embodiments, the cell retention device is operated in alternating tangential flow (ATF) mode.

In some embodiments, the method further includes the steps of: (h) if virus is excreted extracellularly, continuously harvesting excreted virus from the spent media collection vessel; and (i) if virus is produced intracellularly, conducting cell lysis to release intracellular virus and conducting clarification by filtering cell lysate to remove cell debris and/or insoluble microparticles.

In some embodiments, the cell culture system further includes: an input reservoir fluidly connected to the continuous flow bioreactor via an input valve and/or a feed pump, wherein the input reservoir may hold fresh growth media or transfection reagent mixture.

In some embodiments, the bioreactor has a capacity of about 0.015 L to about 2,000 L. In some embodiments, the target VCD has a value of about 1,000,000 cells/mL to about 30,000,000 cells/mL. In some embodiments, wherein the cells comprise a member selected from the group consisting of HEK-293 cells, Hela cells, Vero cells, BHK21 cells and SF9 cells. In some embodiments, the virus comprises adeno-associated viruses (AAV), lentiviruses (LV), retroviruses (RV), adenoviruses (AV), and/or herpes simplex viruses (HSV). In some embodiments, the period of time comprises at least 30 minutes.

In another aspect, the present embodiments are directed to a method of producing viral vectors via cell transfection with continuous addition of transfection reagent mixture at constant volume, including: (a) providing a cell culture system, including: a continuous flow bioreactor, a cell retention device comprising a filtrate port that is fluidly connected to the continuous flow bioreactor; and a spent media collection vessel fluidly connected to the cell retention device; (b) performing cell culture of eukaryotic cells until a viable cell density (VCD) in the bioreactor reaches a target value suitable for transfection; (c) performing transfection of the cells, including continuously feeding transfection reagent mixture at constant bioreactor volume using the feed pump until the transfection reagent mixture is completely transferred to the bioreactor; (d) producing virus in the continuous flow bioreactor by operating the bioreactor under virus production conditions; and (e) if perfusion after transfection is needed, continuously feeding a fresh supply of growth media through the continuous flow bioreactor while the spent media is continuously collected from the permeate of the cell retention device.

In some embodiments, the cell retention device is an ultrafiltration cell retention device. In some embodiments, the cell retention device is operated in tangential flow filtration (TFF) mode. In some embodiments, the cell retention device is operated in altemating tangential flow (ATF) mode.

In some embodiments, the method further includes the steps of: (f) if virus is excreted extracellularly, continuously harvesting excreted virus from the spent media collection vessel; and (g) if virus is produced intracellularly, conducting a cell lysis procedure to release intracellular virus and conducting a clarification procedure by filtering cell lysate to remove cell debris and/or insoluble microparticles.

In some embodiments. the perfusion cell culture system further includes: an input reservoir fluidly connected to the continuous flow bioreactor via an input valve and/or a feed pump, wherein the input reservoir may hold fresh growth media or transfection reagent mixture.

In some embodiments, the bioreactor has a capacity of about 0.015 L to about 2,000 L. In some embodiments, the target VCD has a value of about 1,000,000 cells/mL to about 30,000,000 cells/mL. In some embodiments, the cells comprise a member selected from the group consisting of HEK-293 cells, Hela cells, Vero cells, BHK21 cells and SF9 cells. In some embodiments, the virus comprise adeno-associated viruses (AAV), lentiviruses (LV), retroviruses (RV), adenoviruses (AV), and/or herpes simplex viruses (HSV).

In another aspect, the present embodiments are directed to a method of producing viral vectors via cell transfection with continuous addition of transfection reagent mixture at constant bioreactor volume and continuous harvest of excreted virus during production phase, including: (a) providing a cell culture system, including: a continuous flow bioreactor, a microfiltration cell retention device fluidly connected to the continuous flow bioreactor; an ultrafiltration device fluidly connected to both the microfiltration cell retention device and the continuous flow bioreactor, the ultrafiltration device having a feed port, a retentate port, and a permeate port; and a spent media collection vessel fluidly connected to both the microfiltration cell retention device and the ultrafiltration device; (b) performing cell culture of eukaryotic cells until a viable cell density (VCD) in the continuous flow bioreactor reaches a target value suitable for transfection; (c) performing transfection of the cells, including the steps of: (i) continuously feeding transfection reagent mixture to the continuous flow bioreactor at a constant bioreactor volume for a period of time while continuously feeding the feed port of the ultrafiltration device from the permeate port of the microfiltration cell retention device: (ii) at least partially permeating complexes of DNA and transfection reagents through the microfiltration cell retention device and collecting the complexes at the retentate port of the ultrafiltration device; (iii) redirecting the complexes to the bioreactor; and (iv) repeating steps (i)-(iii) until all the transfection reagent mixture is completely transferred to the continuous flow bioreactor; and (d) producing virus in the continuous flow bioreactor by operating the bioreactor under virus production conditions; and (e) if perfusion after transfection is needed, continuously feeding a fresh supply of growth media through the continuous flow bioreactor while the spent media is continuously collected from the permeate of the microfiltration cell retention device.

In some embodiments, the microfiltration cell retention device is operated in tangential flow filtration (TFF) mode. In some embodiments, the microfiltration cell retention device is operated in alternating tangential flow (ATF) mode.

In some embodiments, the method further comprises the steps of: (f) if virus is excreted extracellularly, continuously harvesting excreted virus from the spent media collection vessel; and (g) if virus is produced intracellularly, conducting a cell lysis procedure to release intracellular virus and conducting a clarification procedure by filtering cell lysate to remove cell debris and/or insoluble microparticles.

In some embodiments, the perfusion cell culture system further includes: an input reservoir fluidly connected to the continuous flow bioreactor via an input valve and/or a feed pump, wherein the input reservoir may hold fresh growth media or transfection reagent mixture.

In some embodiments, the bioreactor has a capacity of about 0.015 L to about 2,000 L. In some embodiments, the target VCD has a value of about 1,000,000 cells/mL to about 30,000,000 cells/mL. In some embodiments, herein the cells comprise a member selected from the group consisting of HEK-293 cells, Hela cells, Vero cells, BHK21 cells, and SF9 cells. In some embodiments, the virus includes adeno-associated viruses (AAV), lentiviruses (LV), retroviruses (RV), adenoviruses (AV), and/or herpes simplex viruses (HSV).

In another aspect, the present embodiments are directed to a method of producing viral vectors via cell transfection with addition of transfection reagent mixture at once, including: (a) providing a cell culture system, including: a continuous flow bioreactor; a first input reservoir fluidly connected to the continuous flow bioreactor, a cell retention device fluidly connected to the continuous flow bioreactor, the cell retention device including a retentate line and a permeate port; a second input reservoir fluidly connected to the retentate line and to the continuous flow bioreactor; and a spent media vessel fluidly connected to the cell retention device; (b) performing cell culture of eukaryotic cells until a viable cell density (VCD) in the continuous flow bioreactor reaches a target value suitable for transfection; (c) performing transfection of the cells, including: continuously feeding transfection reagent mixture contained in the second input reservoir from the retentate line of the cell retention device and to the continuous flow bioreactor; (d) producing virus in the continuous flow bioreactor by operating the bioreactor under virus production conditions; and (e) if perfusion after transfection is needed, continuously feeding a fresh supply of growth media through the continuous flow bioreactor while the spent media is continuously collected from the permeate of the cell retention device.

In some embodiments, the cell retention device is one of a microfiltration device or an ultrafiltration cell retention device. In some embodiments, the cell retention device is operated in tangential flow filtration (TFF) mode. In some embodiments. the cell retention device is operated in alternating tangential flow (ATF) mode.

In some embodiments, the method further includes the steps of: (f) if virus is excreted extracellularly, continuously harvesting excreted virus from the spent media collection vessel: and (g) if virus is produced intracellularly, conducting a cell lysis procedure to release intracellular virus and conducting a clarification procedure by filtering cell lysate to remove cell debris and/or insoluble microparticles.

In some embodiments, the bioreactor has a capacity of about 0.015 L to about 2,000 L. In some embodiments, the target VCD has a value of about 1,000,000 cells/mL to about 30,000,000 cells/mL. In some embodiments, the cells comprise a member selected from the group consisting of HEK-293 cells, Hela cells, Vero cells, BHK21 cells, and SF9 cells. In some embodiments, the virus comprise adeno-associated viruses (AAV), lentiviruses (LV), retroviruses (RV), adenoviruses (AV), and/or herpes simplex viruses (HSV).

In another aspect, the present embodiments are directed to a method of performing cell culture and cell transfection to produce viral vectors, including: providing a cell culture system comprising a bioreactor with an input vessel; providing eukaryotic cells to the bioreactor; expanding the cells within the bioreactor for a period of time such that the growth of the cells is in a log phase until the viable cell density (VCD) reaches a target value; initiating transfection on the cells by disposing a volume of transfection mix within the bioreactor in contact with the cells; performing at least one viral vector production step following transfection.

In some embodiments, initiating transfection includes continuously adding transfection mix to the bioreactor where about 5% to about 95% of the total volume of the bioreactor is filled with cell culture media, and the volume of transfection mix added is about 5% to about 10 times the total volume of the bioreactor. In some embodiments, the target VCD is from about 1,000,000 cells/mL to about 30,000,000 cells/mL. In some embodiments. the method includes providing an ultrafiltration device fluidly coupled downstream of the bioreactor, wherein gradually adding transfection mix comprises adding transfection to the bioreactor mix at a rate approximately equal to a filtrate flow rate of the ultrafiltration device, and wherein gradually adding transfection mix includes adding transfection mix over a period of from about 1 minute to about 30 minutes.

In some embodiments, the method includes concentrating the cells prior to initiating transfection, such that all of the viable cells of the cell culture are contained within a volumetric range from about 5% to about 75% of an interior volume of the bioreactor. In some embodiments, the method includes providing an ultrafiltration device fluidly coupled downstream of the bioreactor, wherein gradually adding transfection mix comprises adding transfection mix to the ultrafiltration cell at a rate faster than a filtrate flow rate of the ultrafiltration device, and wherein gradually adding transfection mix comprises adding transfection mix over a period of from about 1 minute to about 30 minutes.

In some embodiments, the method includes providing at least one of an ultrafiltration device and a microfiltration device fluidly coupled downstream of the bioreactor, the ultrafiltration device including at least one retentate line and at least one permeate line.

In some embodiments, initiating transfection comprises adding transfection mix to the retentate line from a second input vessel. In some embodiments, the at least one viral vector production step comprises operating the bioreactor to culture cells that produce viruses, cell lysis, and/or clarification.

In another aspect, the present embodiments are directed to a cell culture system for producing viral vectors via cell transfection, including: a continuous flow bioreactor; an input reservoir fluidly connected to the continuous flow bioreactor via a first valve and/or a first pump; a cell retention device fluidly connected to the continuous flow bioreactor via a second valve and/or a second pump; and an output reservoir fluidly connected to the cell retention device via a third valve and/or a third pump.

In some embodiments, the cell retention device comprises at least one of a microfiltration cell retention device and an ultrafiltration cell retention device. In some embodiments, the input reservoir contains fresh cell culture media and/or fresh transfection reagent mixture. In some embodiments, the output reservoir collects spent cell culture media or spent transfection reagent mixture.

In some embodiments, the cell retention device is operating in tangential flow filtration (TFF) mode. In some embodiments, wherein the cell retention is operating in alternating tangential flow (ATF) mode.

In some embodiments, each of the first pump, the second pump, and the third pump includes a diaphragm pump, a centrifugal pump, a membrane pump, a peristaltic pump, or a positive displacement pump. In some embodiments, each of the first valve, the second valve, and the third valve includes a disposable valve.

In another aspect, the present embodiments are directed to a cell culture system for producing viral vectors via cell transfection, including: a continuous flow bioreactor; an input reservoir fluidly connected to the continuous flow bioreactor via a first valve and/or a first pump; a microfiltration cell retention device fluidly connected to the continuous flow bioreactor via a second valve and/or a second pump; an ultrafiltration device fluidly connected to both the microfiltration cell retention device and the continuous flow bioreactor via a third valve and/or a third pump; and an output reservoir fluidly connected to both the ultrafiltration device and the third valve.

In some embodiments, the input reservoir contains fresh media or transfection mix. In some embodiments, the output reservoir receives spent media.

In some embodiments, the cell retention device is operating in tangential flow filtration (TFF) mode. In some embodiments, the cell retention device is operating in altemating tangential flow (ATF) mode.

In some embodiments, each of the first pump, the second pump, and the third pump includes a diaphragm pump, a centrifugal pump, a membrane pump, a peristaltic pump, or a positive displacement pump. In some embodiments, each of the first valve, the second valve, and the third valve comprises a disposable valve.

In another aspect, the present embodiments are directed to a cell culture system for producing viral vectors via cell transfection, including: a continuous flow bioreactor; a first input reservoir fluidly connected to the continuous flow bioreactor via a first valve and/or a first pump: a cell retention device fluidly connected to the continuous flow bioreactor via a second valve and/or a second pump, the cell retention device including a retentate line; a second input reservoir fluidly connected to the retentate line and fluidly connected to the continuous flow bioreactor; and an output reservoir fluidly connected to the cell retention device via a third valve and/or a third pump.

In some embodiments, the cell retention device comprises at least one of a microfiltration cell retention device and an ultrafiltration cell retention device. In some embodiments, the first input reservoir contains fresh cell culture media. In some embodiments, the second input reservoir contains fresh transfection reagent mixture. In some embodiments, the output reservoir collects spent cell culture media and/or spent transfection reagent mixture.

In some embodiments, each of the first pump, the second pump, and the third pump includes a diaphragm pump, a centrifugal pump, a membrane pump, a peristaltic pump, or a positive displacement pump. In some embodiments, each of the first valve, the second valve, and the third valve includes a disposable valve.

In some embodiments, the cell retention device operates in tangential flow filtration (TFF) mode. In some embodiments, the cell retention device operates in alternating tangential flow (ATF) mode.

In another aspect, the present embodiments are directed to a cell culture system for producing viral vectors via cell transfection, including: a continuous flow bioreactor; a first input reservoir fluidly connected to the continuous flow bioreactor via a first valve and/or first pump; a cell retention device fluidly connected to the continuous flow bioreactor via a second valve and/or a second pump; an output reservoir fluidly connected to the cell retention device via a third valve and/or third pump, the output reservoir for receiving permeate from the cell retention device; and a second input reservoir fluidly coupled to both the continuous flow bioreactor and the cell retention device.

In some embodiments, the first input reservoir contains fresh cell culture media. In some embodiments, the second input reservoir contains transfection reagent mixture. In some embodiments, the output reservoir collects spent cell growth medium and/or spent transfection reagent mixture.

In some embodiments, the cell retention device includes at least one of a microfiltration cell retention device and an ultrafiltration cell retention device. In some embodiments, the cell retention device operates in tangential flow filtration (TFF) mode. In some embodiments, the cell retention device operates in alternating tangential flow (ATF) mode. In some embodiments, each of the first pump, the second pump, and the third pump includes a diaphragm pump, a centrifugal pump, a membrane pump, a peristaltic pump, and/or a positive displacement pump. In some embodiments, each of the first valve, the second valve, and the third valve comprises a disposable valve.

In some embodiments, the continuous flow bioreactor has a capacity of about 0.015 L to about 2,000 L. In some embodiments, the cell culture medium includes a growth medium configured to culture eukaryotic cells including at least one of HEK-293 cells, HeLa cells, Vero cells, BHK21 cells, and SF9 cells.

In another aspect, the present embodiments are directed to a cell culture system for producing viral vectors via cell transfection, including: a continuous flow bioreactor; a first input reservoir fluidly connected to the continuous flow bioreactor via a first valve and/or first pump; a cell retention device fluidly connected to the continuous flow bioreactor via a second valve and/or a second pump, an output reservoir fluidly connected to the cell retention device via a third valve and/or third pump, the output reservoir for receiving permeate from the cell retention device; a permeate outflow line for removing permeate from the cell retention device, the permeate outflow line fluidly coupling the cell retention device to the output reservoir; and a second input reservoir fluidly coupled to both the continuous flow bioreactor and the permeate outflow line.

In some embodiments, during a transfection phase, permeate flows from the cell retention device to the output reservoir, and when the system is not in transfection phase, permeate flows from the cell retention device to the continuous flow bioreactor via the output reservoir.

In some embodiments, the system further includes a three-way valve disposed within the permeate outflow line between the third pump and the output reservoir, wherein, when the system is in transfection phase, the three-way valve diverts permeate to the output reservoir, and when the system is not in transfection phase, the three-way valve diverts permeate from the cell retention device to the continuous flow bioreactor via the second input reservoir.

In some embodiments, the transfection reagent mixture reservoir is fluidly coupled to the permeate outflow line downstream of the third pump and upstream of the spent media reservoir. In some embodiments, the first input reservoir contains fresh cell culture media. In some embodiments, the second input reservoir contains transfection reagent mixture. In some embodiments, the output reservoir contains spent media.

In another aspect, the present embodiments are directed to a cell culture system for producing viral vectors via cell transfection, including: a continuous flow bioreactor; a cell retention device fluidly connected to the continuous flow bioreactor via a first valve and/or a first pump; an output reservoir fluidly connected to the cell retention device via a second valve and/or a second pump, the spent media reservoir for receiving permeate from the cell retention device, and the second pump including double pump heads, a first flow stream and a second flow stream; an input reservoir for containing at least one of transfection reagent mixture and fresh media, the input reservoir fluidly coupled to the continuous flow bioreactor via the first flow stream: and a permeate outflow line for removing permeate from the cell retention device, the permeate outflow line fluidly coupling the cell retention device to the spent media reservoir via the second flow stream.

In some embodiments, the second pump includes at least a first pump head and a second pump head, wherein the first pump head pumps the first flow stream, and wherein the second pump head pumps the second flow stream. In some embodiments, the second pump comprises at least one of a diaphragm pump, a centrifugal pump, a membrane pump, a peristaltic pump, and a positive displacement pump.

In some embodiments, the first flow stream is fluidly isolated from the second flow stream. In some embodiments, the first flow stream fluidly connects the input reservoir to the continuous flow bioreactor, and wherein the second flow stream fluidly connects the permeate of the cell retention device to the output reservoir. In some embodiments, the output reservoir collects spent cell culture media and/or spent transfection reagent mixture.

### BRIEF DESCRIPTION OF THE DRAWING

The present teachings described herein will be more fully understood from the following description of various illustrative embodiments, when read together with the accompanying drawing. It should be understood that the drawing described below is for illustration purposes only and is not intended to limit the scope of the present teachings in any way.

FIG. 1 is a flow chart showing a conventional transfection method.

FIG. 2 is a schematic diagram of a plot showing viable cell density (VCD) as a function of time of cell culture, and showing different phases of cell growth.

FIG. 3A is schematic diagram of a bioreactor operating at low cell density for transfection.

FIG. 3B is a schematic diagram of a bioreactor operating at high cell density for transfection using perfusion cell culture.

FIG. 3C is a schematic diagram of a bioreactor operating at high cell density for transfection using perfusion cell culture and sub-optimal transfection conditions.

FIG. 4A is a schematic diagram of a configuration of a cell culture system with a cell retention device operated in tangential flow filtration (TFF) mode, according to aspects of the present embodiments.

FIG. 4B is a schematic diagram of a configuration of a cell culture system with a cell retention device operated in alternating tangential flow (ATF) mode, according to aspects of the present embodiments.

FIG. 5 is a schematic diagram of steps in a method using the cell culture system in FIG. 4A, where the cells are concentrated by cell culture volume reduction prior to transfection, according to aspects of the present embodiments.

FIG. 6 is a flow chart showing steps in the method illustrated in FIG. S, where the cells are concentrated by cell culture volume reduction prior to transfection, according to aspects of the present embodiments.

FIG. 7 is a schematic diagram of steps in a transfection method using the cell culture system in FIG. 4A, where the transfection reagent mixture is continuously added at constant bioreactor volume, according to aspects of the present embodiments.

FIG. 8 is a flow chart showing steps in the method illustrated in FIG. 7, where the transfection reagent mixture is continuously added at constant bioreactor volume, according to aspects of the present embodiments.

FIG. 9A is a schematic diagram illustrating a cell culture system with an additional ultrafiltration device connected in series with a microfiltration cell retention device operated in TFF mode to enable the continuous harvesting of excreted virus during the production phase, according to aspects of the present embodiments.

FIG. 9B is a schematic diagram illustrating a cell culture system with an additional ultrafiltration device connected in series with a microfiltration cell retention device operated in ATF mode to enable the continuous harvesting of excreted virus during the production phase, according to aspects of the present embodiments.

FIG. 10 is a flow chart showing steps in a method for using the system in FIG. 9A and/or FIG. 9B, with continuous addition of transfection reagent mixture at constant bioreactor volume and continuous harvest of excreted virus during production phase, according to aspects of the present embodiments.

FIG. 11 is a configuration of a cell culture system to quickly add the transfection mix connected to the retentate of the cell retention device, according to aspects of the present embodiments.

FIG. 12 is a flow chart showing steps in a method for using the cell culture system in FIG. 11, where transfection reagent is added quickly or at once, temporarily increasing total system volume, according to aspects of the present embodiments.

FIG. 13 is a configuration of a cell culture system where a single pump with double pump heads is used to feed media into the bioreactor and to collect spent media from the permeate of the cell retention device, according to aspects of the present embodiments.

FIG. 14 is a configuration of a cell culture system with transfection reagent mixture directly connected to the permeate of the cell retention device, according to aspects of the present embodiments.

FIG. 15 is a plot of production of adeno-associated virus serotype 8 (AAVS) in units of genome copies per liter (GC/L) as a function of days after transfection showing average virus production using cell culture in batch mode (no perfusion) at low VCD, average virus production using cell culture in perfusion mode with a tangential flow depth filtration (TFDF) filter used as cell retention device at high VCD, and virus production using an optimal concentration of transfection reagent using cell culture in perfusion mode with a TFDF filter showing significant increase in yield using the latter process, according to aspects of the present embodiments.

FIG. 16 is a plot of production of adeno-associated virus serotype 9 (AAV9) using a control TFDF perfusion process where the volume of the transfection mix is adjusted to about 10% of the cell culture volume, compared to an optimized TFDF perfusion process without adjusting the volume of transfection mix, in cumulative genome copies per liter (GC/L), showing an approximately 2× increase in AAV9 genomic titer, according to aspects of the present embodiments.

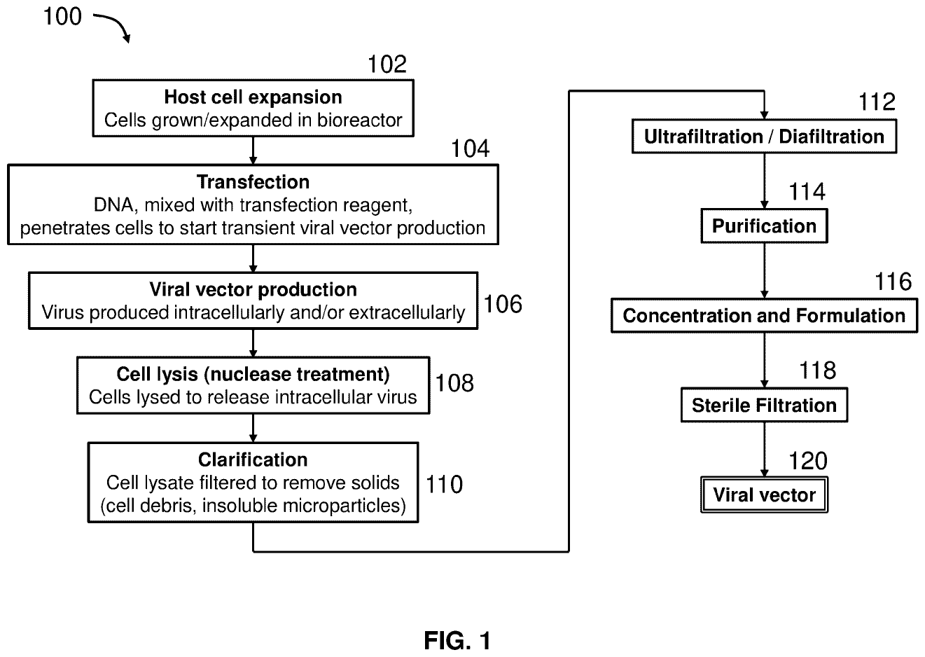

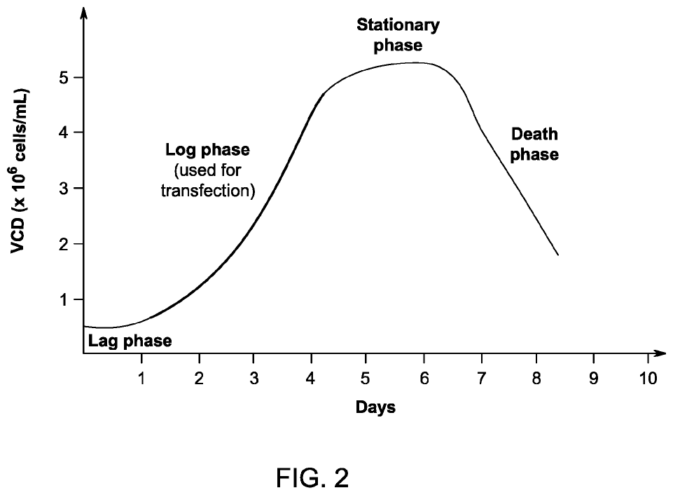

### DEFINITIONS

About, Approximately: As used herein, the terms “about” and “approximately” as used in reference to a value, refers to a value that is similar, in context to the referenced value. In general, those skilled in the art, familiar with the context, will appreciate the relevant degree of variance encompassed by “about” and “approximately” in that context. For example, in some embodiments, the terms “about” and “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adeno-associated virus (AAV): As used herein, the term “adeno-associated virus (AAV)” refers to a category of small viruses that belong to the genus Dependoparvovirus and the family Parvoviridae. AAVs have a single-stranded DNA genome, and typically infect humans and other primates but do not cause disease or strong immune responses, making them suitable for use as viral vectors for gene therapy (i.e., recombinant adeno-associated virus (AAV) vector with engineered genome and/or protein capsid content).

Alternating tangential flow (ATF): As used herein, the term “altemating tangential flow (ATF)” refers to a filtration method for use with a cell retention device for cell culture process intensification. In ATF operations, the direction of flow over the membrane in the cell retention device is alternating.

Associated with: As used herein, the term, “associated with” refers to two events or entities when presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to a disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Bioreactor: As used herein, the term “bioreactor” refers to a device or system for supporting a biologically active environment. In some embodiments, a bioreactor may be a vessel in which chemical processes are carried out involving organisms, cells, biological materials, biochemical agents, and/or chemical agents. In some embodiments, a bioreactor may be classified as batch, fed batch, or continuous.

Cell: As used herein, the term “cell” refers to generally a eukaryotic cell, and within the context of the present disclosures generally refers to a cell that can be cultured in a bioreactor and transfected in order to produce desired biological projects such as viral vectors. In some embodiments, cells may be human cells or animal cells, (e.g., HEK-293 cells (human embryonic kidney cells), Hela cells (immortalized human cervical cancer cells), Vero cells (African green monkey kidney epithelial cells), BHK21 cells (baby hamster kidney cells), or Sf9 cells (Spodoptera frugiperda, or fall army worm, cells)).

Cell culture: As used herein, the term “cell culture” refers to the growth of cells in controlled, artificial conditions outside of their original organisms and/or natural environments. In some embodiments, cell culture requires a cell culture vessel, a growth medium to provide nutrients, an artificial substrate on which cells grow, gases, temperature control, pH control, etc.

Cell culture media or growth media: As used herein, the term “cell culture media” or “growth media” refers to any liquid, or gel, or powder to support cell growth. In some embodiments, cell culture media may be formulated to promote the growth of prokaryotic or eukaryotic cells.

Cell retention device: As used herein. the term “cell retention device” refers to a filtration device designed to maintain cells in a bioreactor while allowing the removal of metabolites and products so that continuous cell culture can occur. In some embodiments, a cell retention device uses membrane filtration. In some embodiments, a cell retention device uses gravitational settling, centrifugation, and acoustic wave separation. In some embodiments, a cell retention device is a microfiltration cell retention device. In some embodiments, a cell retention device is an ultrafiltration cell retention device.

Fed batch cell culture: As used herein, the term “fed batch cell culture” refers to a method of cell culture where there is discrete input of growth medium into a bioreactor and the products are left in the bioreactor.

Gene therapy: As used herein, the term “gene therapy” refers to a technique of treatment or therapy by genetically modifying cells so that a defective or modified genetic sequence is repaired or restored. In some embodiments, gene therapy may be done by using adeno-associated viruses (AAVs) to perform gene insertion.

Microfiltration device or microfiltration cell retention device: As used herein, the term “microfiltration device” or “microfiltration cell retention device” refers to a filtration-based cell retention device, i.e., a cell retention device where the filtration component includes a microfiltration membrane. In some embodiments, a microfiltration membrane may have pore sizes between about 0.1 to 10 μm.

Perfusion cell culture: As used herein, the term “perfusion cell culture” refers to a cell culture method where a cell retention device and continuous media exchange are used to achieve and maintain high cell densities and viabilities over extended periods of time (e.g., weeks).

Tangential flow depth filtration (TFDF): As used herein, the term “tangential flow depth filtration (TFDF)” refers to a mode of filtration combining tangential flow (which has high cell density but poor product passage) with depth filtration (which has low cell density but high product passage) to result in both high cell density and high product passage.

Tangential flow filtration (TFF): As used herein, the term “tangential flow filtration (TFF)” refers to a mode of operation of a filtration system where a fluid passes parallel to a filter membrane (i.e., the flow is tangential to the membrane) to reduce clogging of the filter. In some embodiments, particles larger than the pore size will flow past the membrane as retentate and is recycled back to the feed. In some embodiments, particles smaller than the pore size will pass through and be collected as permeate.

Transfection (TFN): As used herein, the term “transfection (TFN)” refers to a process of artificially introducing nucleic acids (DNA or RNA) into eukaryotic cells. In some embodiments, transfection may be accomplished by physical, chemical, and biological methods.

Transfection mix or transfection reagent mixture: As used herein, the term “transfection mix” or “transfection reagent mixture” refers to a formulation or combination of reagents that are used to induce transfection.

Ultrafiltration device or ultrafiltration cell retention device: As used herein, the term “ultrafiltration device” or “ultrafiltration cell retention device” refers to a filtration-based cell retention device, i.e., a cell retention device where the filtration component includes an ultrafiltration membrane. In some embodiments, ultrafiltration membranes may have pore sizes between about 0.001 and about 0.1 μm.

Viable cell density (VCD): As used herein, the term “viable cell density (VCD)” refers to the number of living cells per unit volume, and is an important indicator of performance in cell culture, that is, the number of cells in a sample volume that are actively growing, dividing or producing a product of interest.

Viral vector: As used herein, the term “viral vector” refers to viruses that have been modified to deliver genetic material into cells. Viral vectors are typically made from viruses that have minimal effect on the target cells. Viral vectors have applications in biomedical research, gene therapy, vaccines, drug delivery, etc.

### Detailed Description of Certain Embodiments

It is contemplated that methods, systems, and processes of the claimed invention encompass variations and adaptations developed using information from the embodiments described herein. Adaptation and/or modification of the methods, systems, and processes described herein may be performed, as contemplated by this description.

Throughout the description, where methods, systems, and processes are described as having, including, or comprising specific components, or where processes and methods are described as having, including, or comprising specific steps, it is contemplated that, additionally, there are systems of the present invention that consist essentially of, or consist of, the recited components, and that there are processes and methods according to the present invention that consist essentially of, or consist of, the recited steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader; the presence and/or placement of a header is not intended to limit the scope of the subject matter described herein.

The present disclosure provides improved technologies for the production of viral vectors via optimized cell transfection at high viable cell densities.

### A. Configurations of Systems

The present disclosure provides several configurations of cell culture systems that may be used for the production of viral vectors via transfection. Each of the configurations described below include a perfusion cell culture system and one or more cell retention devices and/or filtration devices, along with various modes of introducing transfection reagent mixtures. It is contemplated that options for operating conditions, dimensions, flow rates, components, etc., described in each configuration below may be applicable to other configurations or combinations.

In addition to enabling transfection at high VCD using optimal concentrations of reagents, all configurations described using a microfiltration cell retention device can be used to perform an integrated clarification, with or without cell lysis, to recover the virus remaining in the bioreactor at the end of the virus production phase.

For all configurations described herein, entirely closed and disposable flow path assemblies are envisioned so that all operations can be conducted without opening the process and risking any contamination. For example, the operation to change from cell culture media to transfection reagent mixture should be conducted by operating disposable valves to connect the necessary vessel to the system.

### 1. Cell Culture System With Cell Retention Device

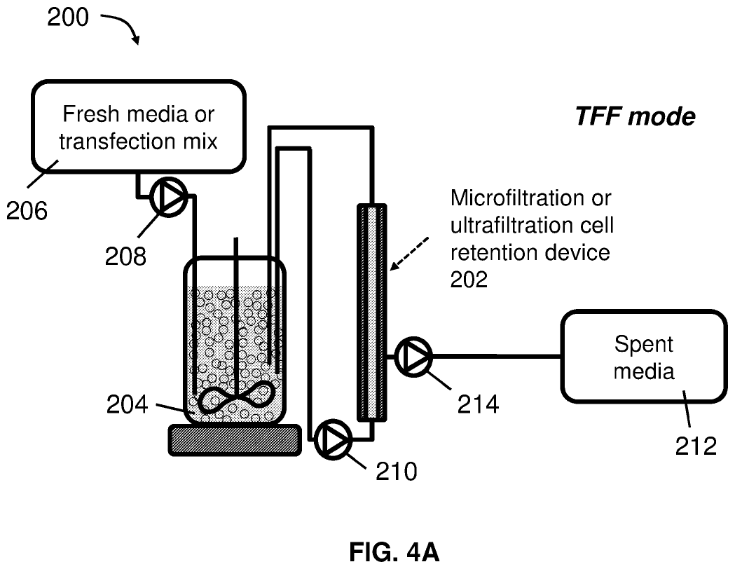

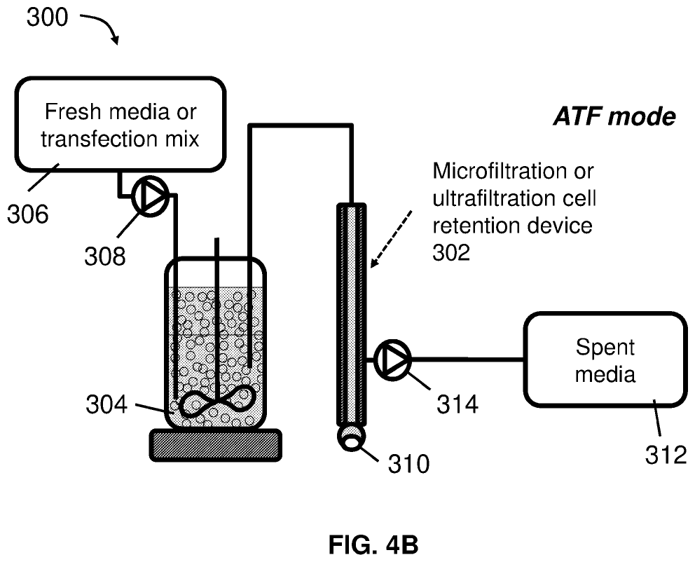

The present disclosures describe a cell culture system that includes a cell retention device (i.e., either a microfiltration cell retention device or an ultrafiltration cell retention device). FIG. 4A is a schematic diagram of a configuration of a cell culture system 200 with a cell retention device 202 operated in tangential flow filtration (TFF) mode, according to aspects of the present embodiments. FIG. 4B is a schematic diagram of a configuration of a cell culture system 300 with a cell retention device 302 operated in altemating tangential flow (ATF) mode, according to aspects of the present embodiments.

In some embodiments, the configurations of FIGS. 4A and 4B may generally include a continuous flow bioreactor (204 or 304); an input reservoir (e.g., for holding fresh media or transfection mix) (206 or 306) fluidly connected to the continuous flow bioreactor via a first valve and/or a first pump (208 or 308); a cell retention device (202 or 302) (e.g., a microfiltration cell retention device or an ultrafiltration cell retention device) fluidly connected to the continuous bioreactor via a second valve and/or a second pump 210 (for the TFF mode, 310 for the ATF mode); and an output reservoir (212 or 312) (e.g., for collecting spent media) fluidly connected to the cell retention device via a third valve and/or a third pump (214 or 314).

In some embodiments, if the cell retention device is operating in ATF mode, the second pump is not fluidly connected to the continuous flow bioreactor but only operates at the cell retention device to provide alternating vacuum pressure. In some embodiments, the output reservoir is fluidly connected to the permeate of the cell retention device to collect components that have passed through the filter membrane. In some embodiments, each of the pumps may be a diaphragm pump, a centrifugal pump, a membrane pump, a peristaltic pump, and/or a positive displacement pump. In some embodiments, each of the valves may be a disposable valve in order to minimize contamination.

### 2. Cell Culture System With Microfiltration Cell Retention Device and Ultrafiltration Device

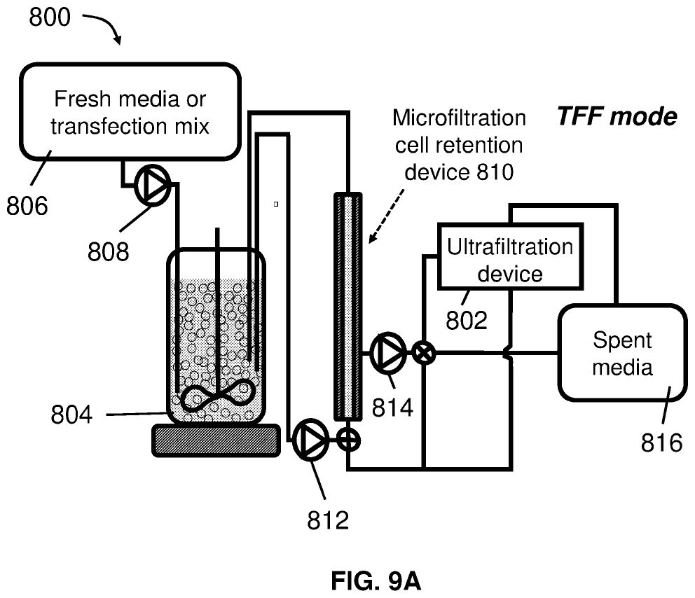

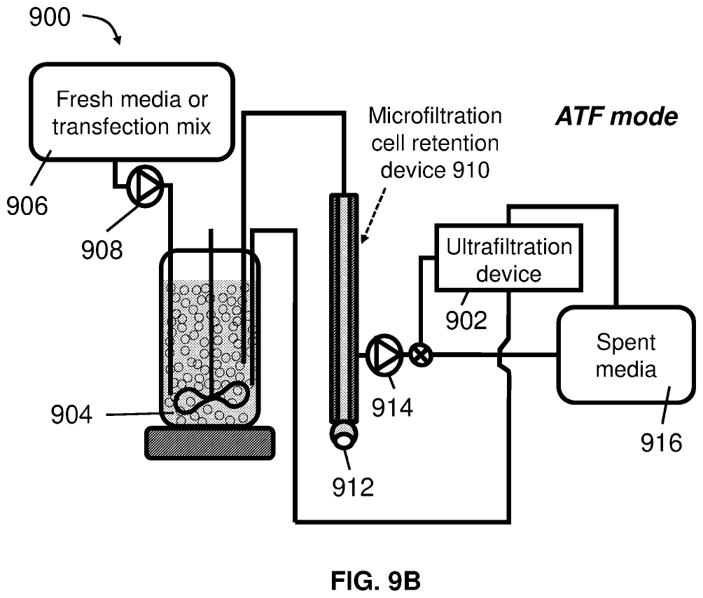

The present disclosure describes a cell culture system that includes both a microfiltration cell retention device and an ultrafiltration cell retention device. FIG. 9A is a cell culture system 800 with an additional ultrafiltration device 802 connected in series with a microfiltration cell retention device operated in TFF mode to enable the continuous harvesting of excreted virus during the production phase, according to aspects of the present embodiments. FIG. 9B is a cell culture system with an additional ultrafiltration device 902 connected in series with a microfiltration cell retention device operated in ATF mode to enable the continuous harvesting of excreted virus during the production phase, according to aspects of the present embodiments.

In some embodiments, the configurations shown in FIG. 9A and FIG. 9B may generally include a continuous flow bioreactor (804 or 904); an input reservoir (806 or 906) (e.g., for holding fresh media or transfection reaction mixture) fluidly connected to the continuous flow bioreactor via a first valve and/or a first pump (808 or 908); a microfiltration cell retention device (810 or 910) fluidly connected to the continuous flow bioreactor via a second valve and/or a second pump 812 for the TFF mode or pump 912 for altemating flow in ATF mode, an ultrafiltration device (802 or 902) fluidly connected to both the microfiltration cell retention device (810 or 910) and the continuous flow bioreactor (804 or 904) via a third valve and/or a third pump (814 or 914); and an output reservoir (816 or 916) (e.g., for collecting spent media) fluidly connected to both the ultrafiltration device (802 or 902) and the third valve and/or pump (814 or 914).

In some embodiments, the second valve and/or second pump are fluidly connected to a feed port of the microfiltration cell retention device. In some embodiments, the third valve and/or third pump are fluidly connected to a permeate port of the microfiltration cell retention device. In some embodiments, where the microfiltration device is operating in ATF mode, the second pump operates on the microfiltration cell retention device but is not fluidly connected to another portion of the system. In some embodiments, each pump may be a diaphragm pump, a centrifugal pump, a membrane pump, a peristaltic pump, and/or a positive displacement pump. In some embodiments, each valve may be a disposable valve to minimize contamination.

### 3. Cell Culture System With Transfection Reagent Mixture Reservoir Connected to Retentate Line of Cell Retention Device

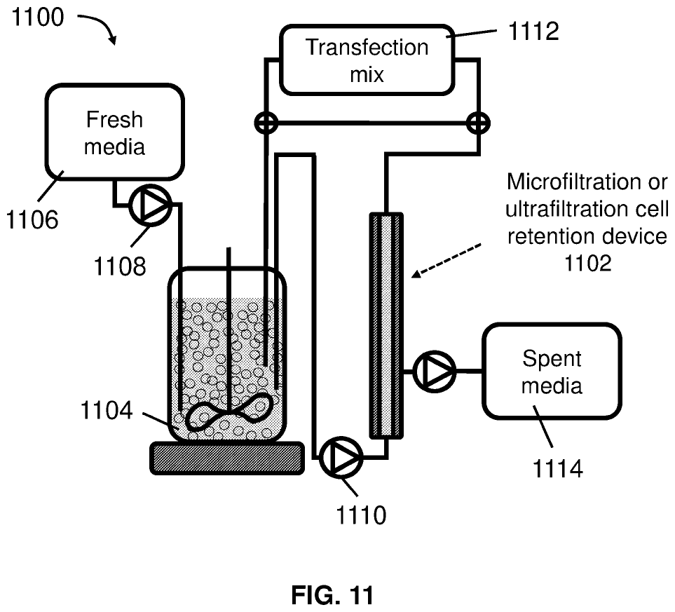

The present disclosure describes a cell culture system that includes a transfection reagent mixture reservoir connected to a retentate line of the cell retention device. FIG. 11 is a configuration of a cell culture system 1100 to quickly add the transfection mix connected to the retentate of the cell retention device 1102, according to aspects of the present embodiments.

In some embodiments, the configuration shown in FIG. 11 may generally include a continuous flow bioreactor 1104; a first input reservoir 1106 (e.g., to hold fresh cell culture media) fluidly connected to the continuous flow bioreactor 1104 via a first valve and/or a first pump 1108, a cell retention device 1102 (e.g., a microfiltration cell retention device or an ultrafiltration cell retention device) fluidly connected to the continuous flow bioreactor 1104 via a second valve and/or a second pump 1110; a second input reservoir 1112 (e.g., to hold transfection reagent mixture) fluidly connected to a retentate line of the cell retention device 1102 and fluidly connected to the continuous flow bioreactor 1104; and an output reservoir 1114 (e.g., to hold spent media) fluidly connected to the cell retention device via a third valve and/or a third pump. In some embodiments, each of the pumps may be a diaphragm pump, a centrifugal pump, a membrane pump, a peristaltic pump, and/or a positive displacement pump. In some embodiments, each of the valves may be a disposable valve to minimize contamination. In some embodiments, the third valve and/or third pump are fluidly connected between a permeate port of the cell retention device and the output reservoir.

### 4. Cell Culture System With Single Pump With Double Pump Heads to Feed Media Into Bioreactor and Collect Spent Media From Permeate of Cell Retention Device

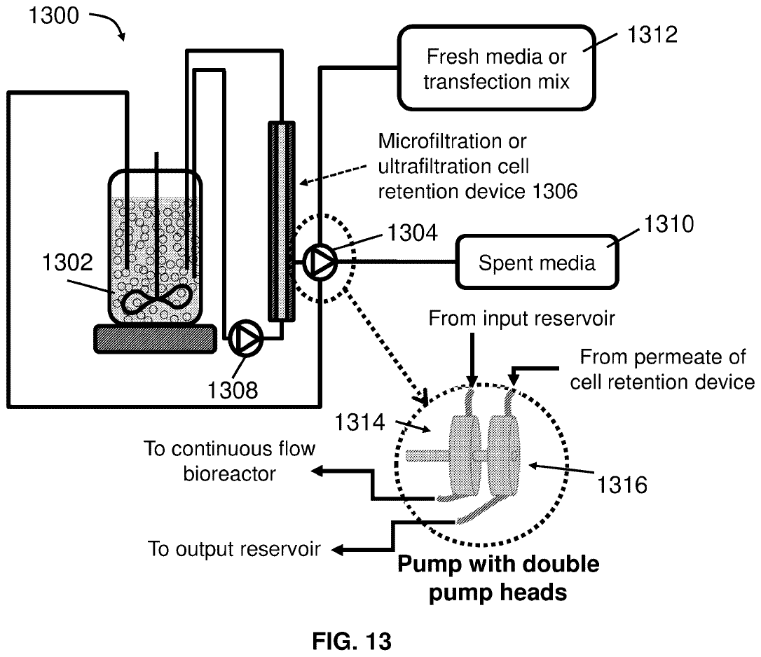

The present disclosures describe a cell culture system that includes a single pump with double pump heads. FIG. 13 is a configuration of a cell culture system 1300 where a single pump 1304 with double pump heads is used to feed media into the bioreactor 1302 and to collect spent media from the permeate of the cell retention device 1306, according to aspects of the present embodiments.

In some embodiments, the configuration shown in FIG. 13 may generally include a continuous flow bioreactor 1302; a cell retention device 1306 (e.g., a microfiltration cell retention device or an ultrafiltration cell retention device) fluidly connected to the continuous flow bioreactor 1302 via a first valve and/or a first pump 1308; an output reservoir 1310 (e.g., for collecting spent cell culture media and/or spent transfection reagent mixture) fluidly connected to the cell retention device 1306 via a second valve and/or a second pump 1304, the output reservoir for receiving permeate from the cell retention device 1306, and the second pump 1304 comprising double pump heads, a first flow stream and a second flow stream; an input reservoir 1312 for containing at least one of transfection reagent mixture and fresh media, the input reservoir fluidly coupled to the continuous flow bioreactor 1302 via the first flow stream 1314; and a permeate outflow line for removing permeate from the cell retention device, the permeate outflow line fluidly coupling the cell retention device to the spent media reservoir via the second flow stream 1316.

In some embodiments, the configuration shown in FIG. 13 may have a second pump including a first pump head that pumps the first flow stream, and a second pump head that pumps the second flow stream. In some embodiments, the first pump head and the second pump head are coaxially aligned (or concentrically disposed about a central axis or centerline), or are arranged adjacent to each other without a common central axis. In some embodiments, the pumps may be at least one of a diaphragm pump, a centrifugal pump, a membrane pump, a peristaltic pump, and/or a positive displacement pump. In some embodiments, the first flow stream is fluidly isolated from the second flow stream. In some embodiments, the first flow stream fluidly connects the input reservoir to the continuous flow bioreactor, and the second flow stream fluidly connects the permeate of the cell retention device to the output reservoir.

### 5. Cell Culture System With Single Pump With Transfection Reagent Mixture Directly Connected to Permeate of Cell Retention Device

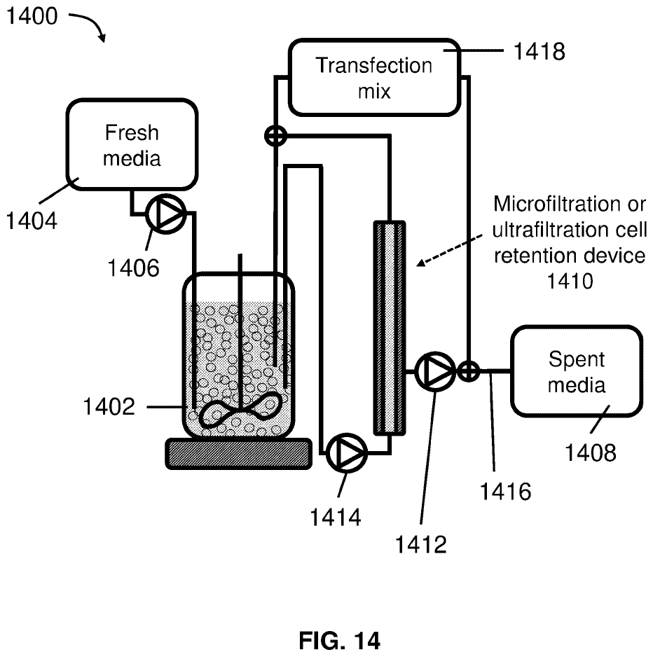

The present disclosure describes a cell culture system with transfection reagent mixture directly connected to the permeate of the cell retention device. FIG. 14 is a configuration 1400 of a cell culture system with transfection reagent mixture directly connected to the permeate of the cell retention device 1410, according to aspects of the present embodiments.

In some embodiments, the configuration 1400 shown in FIG. 14 may generally include a continuous flow bioreactor 1402; an input reservoir 1404 (e.g., containing fresh cell growth media) fluidly connected to the continuous flow bioreactor 1402 via a first valve and/or a first pump 1406; a cell retention device 1410 fluidly connected to the bioreactor 1402 via a second valve and/or a second pump 1414; an output reservoir 1408 (e.g., to collect spent media) fluidly connected to the cell retention device 1410 via a second valve and/or second pump 1412, the output reservoir for receiving permeate from the cell retention device; a permeate outflow line for removing permeate from the cell retention device, the permeate outflow line fluidly coupling the cell retention device 1410 to the output reservoir 1408; and a second input reservoir 1418 (e.g., containing transfection reagent mixture) fluidly coupled to both the continuous flow bioreactor 1402 and the permeate outflow line 1416.

In some embodiments, during a transfection phase, permeate flows from the cell retention device to the output reservoir. In some embodiments, when the system is not operating in a transfection phase, permeate flows from the cell retention device to the continuous flow bioreactor via the output reservoir. In some embodiments, a three-way valve is disposed within the permeate outflow line between the third pump and the output reservoir. When the system is in transfection phase, the three-way valve diverts permeate to the output reservoir, and when the system is not operating in transfection phase, the three-way valve diverts permeate from the cell retention device to the continuous flow bioreactor via the second input reservoir.

### B. Methods of Operation of Systems

The present embodiments include several methods of operating the various configurations of systems described above. The methods described below enable the production of viral vectors at optimized higher values of viable cell density.

### 1. Concentrate Cells Before Transfection

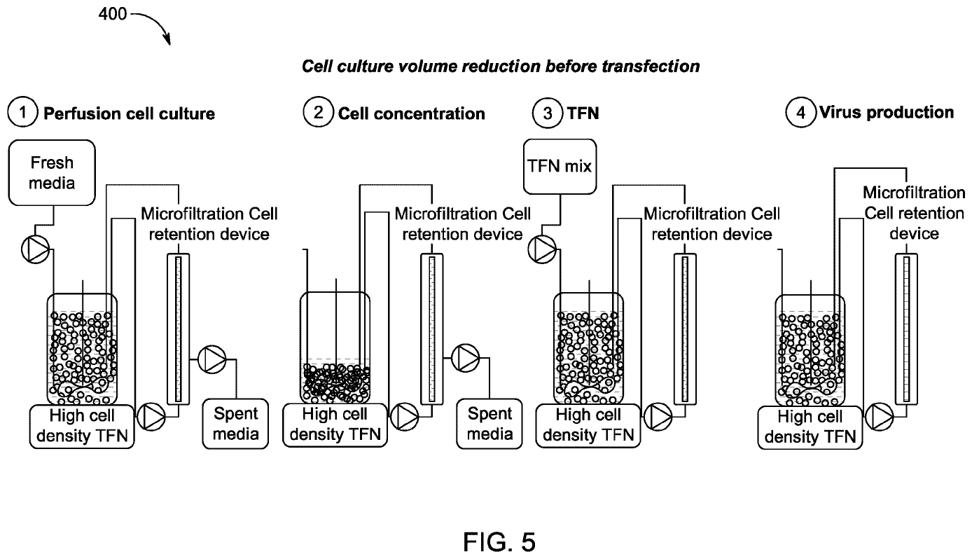

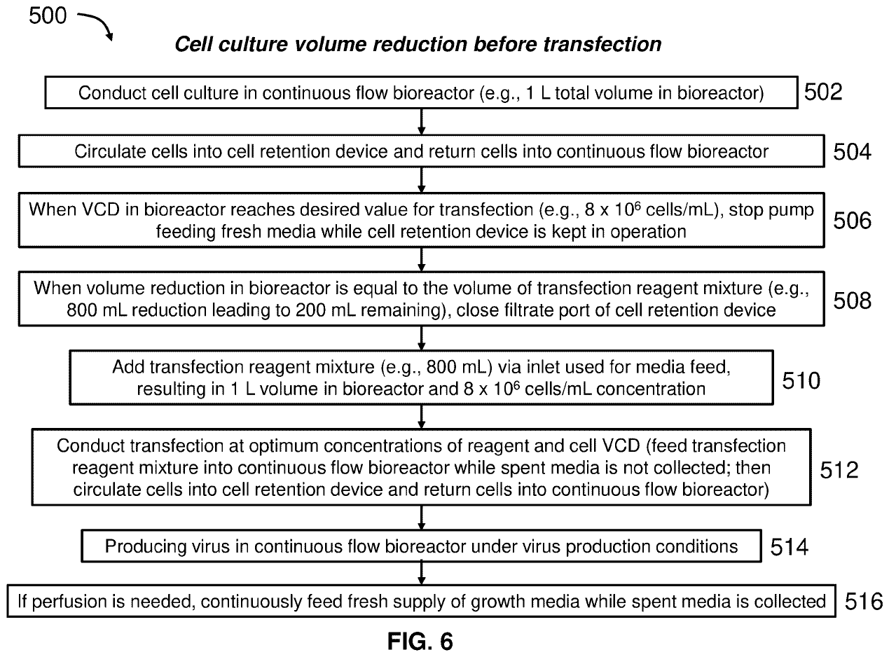

In some embodiments, the present disclosure includes a method of performing transfection where the cells are concentrated before transfection. FIG. 5 is a schematic diagram of steps in a method using the cell culture system in FIG. 4A, where the cells are concentrated prior to transfection, according to aspects of the present embodiments. FIG. 6 is a flow chart showing steps in the method 500 illustrated in FIG. 5, according to aspects of the present embodiments. In this method 500, the concentration of cells is accomplished by reducing the cell culture volume.

This method 500 may generally include the steps of: conducting cell culture in a continuous flow bioreactor (step 502); circulating cells into a cell retention device, returning cells into the continuous flow bioreactor until the viable cell density (VCD) in the bioreactor (step 504); when the VCD reaches a desired value for transfection, stopping the pump feeding the fresh media while the cell retention device is kept in operation (step 506); closing the filtrate port of the cell retention device is when the reduction in volume in the reactor is equal to the volume of transfection reagent mixture (step 508); adding transfection reagent mixture via the same inlet used for media feed, resulting in the same original bioreactor volume (step 510); conducting transfection at optimum concentrations of reagent and cell VCD (which includes feeding transfection reagent mixture into the continuous flow bioreactor while spent media is not collected, circulating cells into the cell retention device, and returning cells into the continuous flow bioreactor (step 512); producing virus in the continuous flow bioreactor under virus production conditions (step 514); and, if perfusion is needed after transfection, continuously feeding a fresh supply of growth media while spent media is collected (step 516).

### 2. Continuously Add Transfection Reagent Mixture at Constant Bioreactor Volume

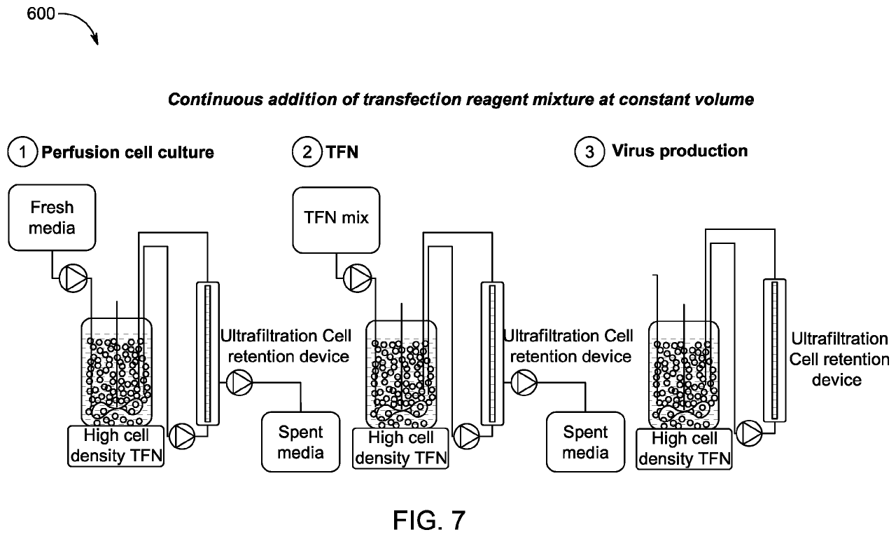

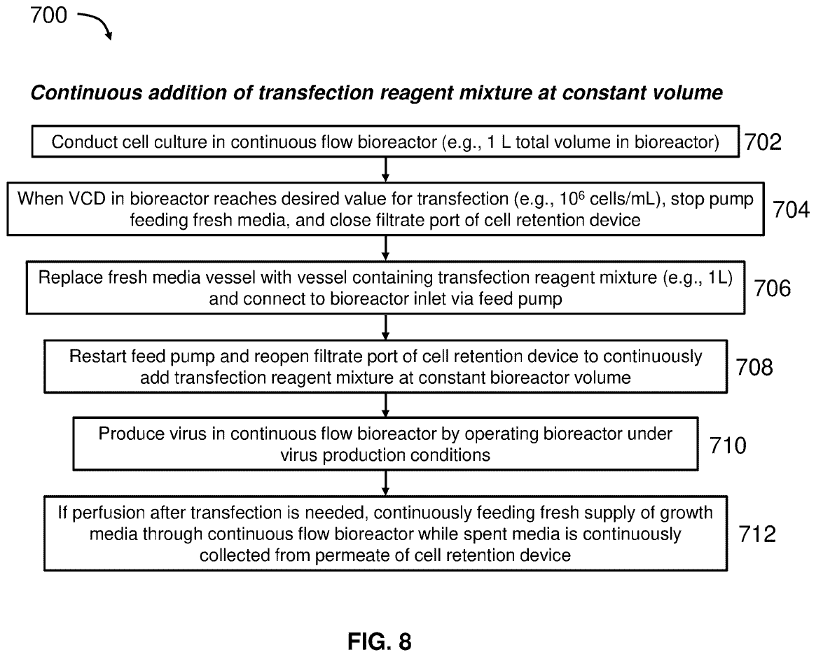

In some embodiments, the present disclosure includes a method of adding transfection reagent mixture at a constant bioreactor volume. FIG. 7 is a schematic diagram of steps in a transfection method using the cell culture system in FIG. 4A, where the transfection reagent mixture is continuously added at constant bioreactor volume, according to aspects of the present embodiments. FIG. 8 is a flow chart showing steps in the method 700 illustrated in FIG. 7, according to aspects of the present embodiments.

This method 700 may generally include the steps of: conducting cell culture in a continuous flow bioreactor (step 702): when the VCD reaches a desired value for transfection, the pump feeding fresh media is stopped and the filtrate port of the cell retention device is closed (step 704); replacing the fresh media vessel with a vessel containing transfection reagent mixture and connecting it to the bioreactor inlet via the feed pump (step 706); restarting the feed pump and reopening the filtrate port of the cell retention device to continuously add transfection reagent mixture at constant bioreactor volume (step 708): producing virus in the continuous flow bioreactor by operating the bioreactor under virus production conditions (step 710); and, if perfusion after transfection is needed, continuously feeding a fresh supply of growth media through the continuous flow bioreactor while spent media is continuously collected from the permeate of the cell retention device (step 712).

### 3. Quickly and Continuously Add Transfection Reagent Mixture at Constant Bioreactor Volume

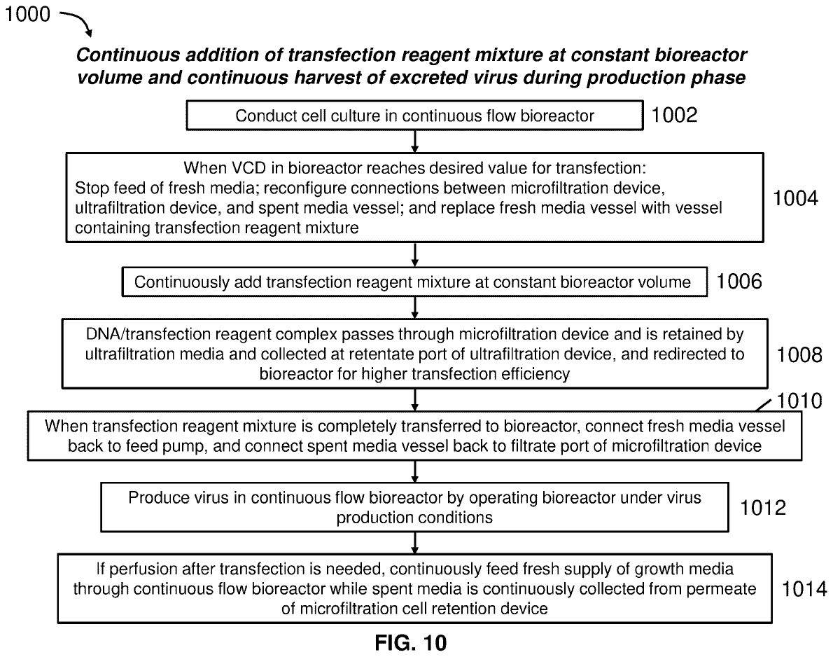

In some embodiments, the present disclosure includes a method of quickly and continuously adding transfection reagent mixture at constant bioreactor volume. FIG. 9 is a schematic diagram of steps in a transfection method using the cell culture system in FIG. 4A, where the transfection reagent mixture may be quickly and continuously added at constant bioreactor volume, and an ultrafiltration cell retention device is used, according to aspects of the present embodiments. FIG. 10 is a flow chart showing steps in the method 1000 illustrated in FIG. 9, according to aspects of the present embodiments.

This method 1000 may generally include the steps of: conducting cell culture in a continuous flow bioreactor (step 1002); when the VCD reaches a desired value for transfection, at which the feed of fresh media is stopped, connections between the microfiltration device, ultrafiltration device, and spent media vessel are reconfigured, and the fresh media vessel is replaced with a vessel containing transfection reagent mixture (step 1004); continuously adding transfection reagent mixture at constant bioreactor volume (step 1006); allowing the DNA/transfection reagent complex to pass through the microfiltration device, retaining it by the ultrafiltration media, collecting it at the retentate port of the ultrafiltration device, and redirecting it to the bioreactor for higher transfection efficiency (step 1008); (1) connecting the fresh media vessel back to the feed pump, and (2) connecting the filtrate port of the microfiltration device back to the spent media vessel when transfection reagent mixture is completely transferred to bioreactor (step 1010); producing virus in the continuous flow bioreactor by operating the bioreactor under virus production conditions (step 1012); and, if perfusion after transfection is needed, continuously feeding a fresh supply of growth media through the continuous flow bioreactor while spent media is continuously collected from the permeate of the microfiltration cell retention device (step 1014).

### 4. Quickly Add Transfection Reagent While Temporarily Increasing Total System Volume

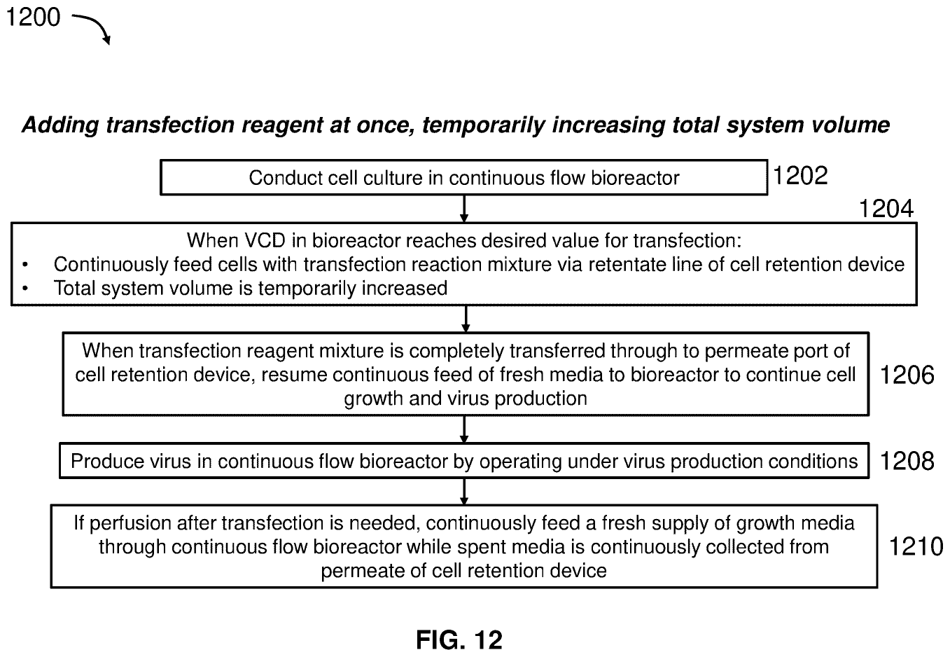

In some embodiments, the present disclosure includes a method of quickly adding transfection reagent while temporarily increasing total system volume. FIG. 12 is a flow chart showing steps in a method 1200 for using the cell culture system in FIG. 11, according to aspects of the present embodiments.

The method 1200 may generally include the steps of: conducting cell culture in a continuous flow bioreactor (step 1202); when the VCD reaches a desired value for transfection, performing transfection by continuously feeding cells with transfection reagent mixture via the retentate line of the cell retention device, and thus temporarily increasing the total system volume (step 1204); resuming continuous feed of fresh media to the bioreactor to continue cell growth and virus production when the transfection reagent mixture is completely transferred through to the permeate port of the cell retention device (step 1206); producing virus in the continuous flow bioreactor by operating under virus production conditions (step 1208); and, if perfusion after transfection is needed, continuously feeding a fresh supply of growth media through the continuous flow bioreactor while spent media is continuously collected from the permeate of the cell retention device (step 1210).

### 5. Infection-Based Process

Virus production from cell culture can also be achieved by infecting cells with a virus stock solution instead of using a transfection method, in accordance with aspects of the present embodiments. The virus production method of the present embodiments can be used for viral vaccine production. According to the disclosed process, virus particles are added to the cell culture to infect cells and deliver genetic material required to initiate the virus production. Each of the infected cells produce more virus particles, which are then released in the cell culture media.

When transfection is used, the process efficiency is highly dependent on the amount of DNA and transfection reagent used versus the number of cells to be transfected. Similarly, using virus infection, the process efficiency is highly dependent on the amount of virus particles used versus the number of cells to be infected. The ratio between the number of virus particles and the number of cells is called multiplicity of infection (MOD). Optimal MOI is defined during process development to maximize virus production.

Process intensification using perfusion cell culture allows for infection at high cell density, and therefore produces more virus post infection. To keep the MOI at an optimum value, an increased number of virus particles is needed as more cells are to be infected. A higher volume of virus stock solution is therefore needed, but will not necessarily fit in the available volume inside the bioreactor vessel.

To allow for keeping the MOI at optimal value by adding a higher volume of virus stock solution, all embodiments related to transfection can be used for virus infection at high cell density. For example, according to aspects of the present embodiments, virus infection is initiated in the first 2-3 steps of the methods described in FIGS. 6, 8, 10, and 12 (for instance, in place of transfection). In addition, at steps 506, 706, 1004, and 1204 of FIGS. 6, 8, 10, and 12 respectively, action is taken once a desired level of cell infection (that is, rather than transfection) has occurred.

### EXAMPLES

### Example 1: Transfection Method for Batch Cell Culture

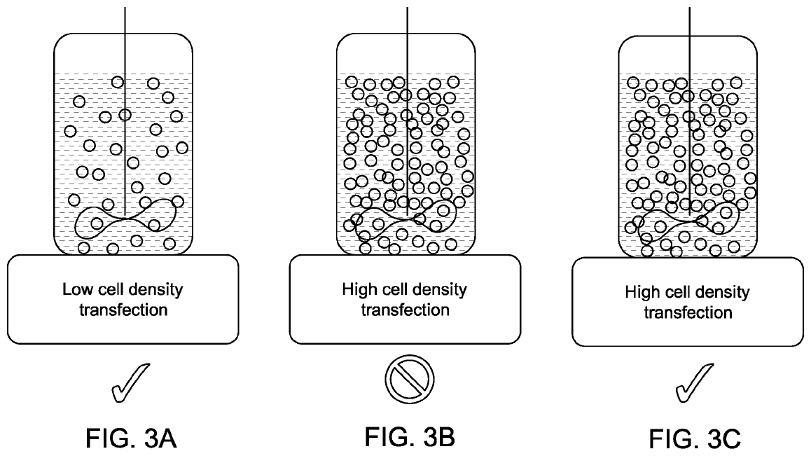

The present example describes cell culture and transfection according to the system illustrated in FIG. 3A. In this example, cell culture is at a relatively low cell concentration. Cell culture is performed here in a 1 L volume bioreactor vessel containing cells grown at 1×10⁶ cells/mL. At this value of viable cell density, transfection requires 100 mL of transfection reagent mixture to be added to 900 mL of cell culture. If 1 μg of DNA is added at 1×10⁶ cells/mL, there is 1 mg of DNA (or 1 mL at 1 mg/mL). A concentration 1 μg of DNA in 1 μL transfection reagent then requires 1 mL of transfection reagent. Thus, the concentration of transfection reagent is 10 μL/mL, or 100 mL of transfection mixture being added to 900 mL of cell culture. This example illustrates some feasible concentrations and volumes for transfection at a relatively low cell concentration.

### Example 2: Transfection Method for Perfusion Cell Culture

The present example describes cell culture and transfection according to the system illustrated in FIG. 3B. In this example, cell culture is at a relatively high cell concentration. Cell culture is performed here in a 1 L volume bioreactor vessel containing cells grown at 10×10°cells/mL (i.e., 10× higher than the concentration in Example 2) in a perfusion cell culture system, which enables cells to stay in the log phase growth at a higher cell density. At this value of viable cell density, transfection requires 1000 mL of transfection reagent mixture to be added in order to keep all transfection parameters at optimum values, which is not feasible in a 1 L bioreactor vessel. Thus, additional technologies are needed to enable transfection at higher cell concentrations, as described in the embodiments disclosed herein.

### Example 3: Transfection Method for Perfusion Cell Culture With Sub-Optimal Transfection Conditions

The present example describes cell culture and transfection according to the system illustrated in FIG. 3C. In this example, for transfection at high VCD values in bioreactor vessels, a compromise must be made to use sub-optimal transfection conditions. For example, the total volume of transfection mix can be reduced by increasing the concentration of the transfection reagent to the free volume available for the cell culture. This leads to (i) low transfection efficiency due to the cytotoxicity of transfection reagent, and (ii) the bioreactor not being used at full capacity (e.g., less than 700 ml of cell culture volume only). In this example, cell culture is at a relatively high cell concentration. Cell culture is performed here in a 1 L volume bioreactor vessel containing cells grown at a relatively high concentration of 10×10⁶ cells/mL in a perfusion cell culture system. If 1 μg of DNA per 1×10⁶ cells is added, there will be 10 mg DNA, or 1 mL at 1 mg/mL. The DNA/transfection reagent ratio is 1, so 10 mg of transfection reagent is needed. The concentration of transfection reagent in the transfection mix is then 30 μL/mL, and 333 mL transfection volume will be added to 667 mL of cell culture volume. This arrangement will lead to low transfection efficiency due to the cytotoxicity of transfection reagent, as well as the bioreactor volume not being used at full capacity. Thus, this example also illustrates that additional technologies are needed to enable transfection at higher cell concentrations and optimized transfection conditions, as described in the embodiments disclosed herein.

### Example 4: Production of AAVS Using Optimized TFDF-Based High VCD Transfection

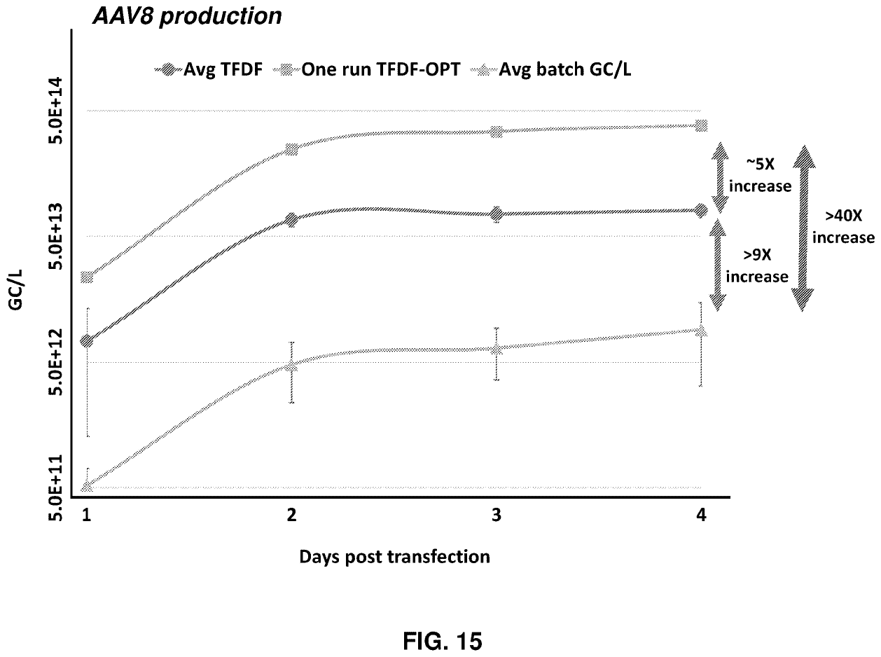

The present example describes experimental results showing the production of adeno-associated virus serotype 8 (AAV8) using three different methods: conventional batch reactor transfection, tangential flow depth filtration (TFDF) transfection, and optimized TFDF transfection. TFDF is similar to TFF, but also combines a depth filter, resulting in both high cell density and high product passage. FIG. 15 is a plot of production of adeno-associated virus serotype 8 (AAV8) in units of genome copies per liter (GC/L) as a function of days after transfection showing average virus production using cell culture in batch mode (no perfusion) at low VCD, average virus production using cell culture in perfusion mode with a tangential flow depth filtration (TFDF) filter used as cell retention device at high VCD, and virus production using an optimal concentration of transfection reagent using cell culture in perfusion mode with a TFDF filter showing significant increase in yield using the latter process, according to aspects of the present embodiments.

In the experiments shown in FIG. 15, the data showing batch mode virus production is the average of three cell culture runs that used a transfection mix containing the transfection reagent, with a volume of 10% of the cell culture volume, or about 0.2 L, and a low VCD value of about 3×10⁶ cells/mL. The transfection mixture was added to the cell culture volume of about 2 L.

Still referring to FIG. 15, the data showing TFDF was the average of three cell culture runs in perfusion mode, with a high VCD of about 9×10⁶ cells/mL using about the same volume of cell culture (about 2 L). The amount of transfection reagent to be added is dependent on the number of cells. Since there are about 3 times more cells in the bioreactor vessel with these perfusion cell culture runs (i.e., VCD of about 9×10⁶ cells/mL) compared to the batch runs, about 3 times more transfection reagent is needed in the transfection mix to be added to the cell culture. To keep the concentration of the transfection reagent the same than for batch runs, the volume of transfection mix containing the transfection reagent should be 3 times higher (i.e., about 0.6 L vs. about 0.2 L for batch runs). However, the volume available in the bioreactor vessel is not enough to add about 0.6 L, and is therefore limited to about 10% of the cell culture volume (i.e., about 0.2 L for every 2L cell culture volume). The final concentration of the transfection reagent is consequently increased by a factor of about 3 and is outside of the optimal concentration range.

FIG. 15 also shows data marked “TFDF-OPT”, referring to an optimal concentration of transfection reagent used, i.e., about 3 times higher volume of transfection mix volume is added to the bioreactor vessel (i.e., about 0.6 L for every 2 L cell culture volume at about 9×10⁶ cells/ml VCD) compared to the previous batch and not-optimized TFDF runs (where there was about 0.2 L transfection mix added to every 2 L cell culture volume). The addition of the larger volume in the bioreactor vessel is enabled by practicing the methods and systems of the current embodiments.

These results show that there is about a greater than 9-fold increase in yield of AAV 8 at 4 days after transfection for the average of TFDF-based transfection compared to batch reactor transfection, and a further 5-fold increase above the average TFDF results for a single run of optimized TFDF transfection. Overall, there is a greater than 40-fold increase in AAV8 yield for an optimized TFDF-based transfection over average batch transfection.

These results indicate that optimizing the transfection process can produce significant increases in the yield of viral vector production.

### Example 5: Production of AAV9 Using Optimized Transfection for TFDF-Based Perfusion Cell Culture

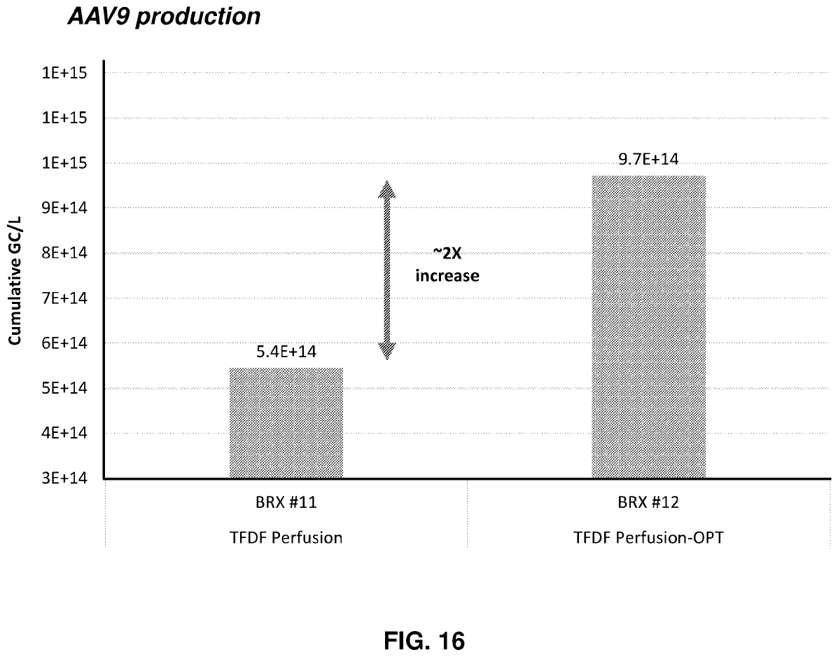

The present example describes experimental results showing the production of adeno-associated virus serotype 9 (AAV9) using different processes. FIG. 16 is a plot of production of adeno-associated virus serotype 9 (AAV9) using a control TFDF perfusion process where the volume of the transfection mix is adjusted to about 10% of the cell culture volume, compared to an optimized TFDF perfusion process without adjusting the volume of transfection mix, in cumulative genome copies per liter (GC/L), showing an approximately 2× increase in AAV 9 genomic titer, according to aspects of the present embodiments. These results show two experiments for producing AAV9. The bar on the left shows the cumulative yield of virus copies for a TFDF perfusion process, while the bar on the right shows the cumulative yield of virus copies for an optimized TFDF perfusion process. There is about a 2-fold increase in AAV9 yield for the optimized procedure where the concentration of the transfection reagent in the transfection mix is optimized. The labels BRX refer to separate bioreactor run numbers.

Referring still to FIG. 16, the cell cultures both used perfusion cell culture with a TFDF as the cell retention device to enable high VCD at transfection (about 9×10⁶ cells/mL). For the control run (labelled “TFDF Perfusion”), the volume of the transfection mix is adjusted to about 10% (i.e., about 0.2 L) of the cell culture volume (i.e., about 2 L) to fit in the bioreactor vessel, consequently increasing the concentration of the transfection reagent outside of the optimal range. In the optimized run (labelled “TFDF Perfusion-OPT”) the run is conducted at the same VCD of about 9×10⁶ cells/mL as the previous run, but without adjusting the volume of transfection mix, in this case about 30% or about 0.6 L of the cell culture volume (i.e., about 2 L).

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed:

1. A cell culture system for producing viral vectors via cell transfection, comprising:
a continuous flow bioreactor;
a first input reservoir fluidly connected to the continuous flow bioreactor via a first valve and/or first pump;
a cell retention device fluidly connected to the continuous flow bioreactor via a second valve and/or a second pump;
an output reservoir fluidly connected to the cell retention device via a third valve and/or third pump, the output reservoir for receiving permeate from the cell retention device; and
a second input reservoir fluidly coupled to both the continuous flow bioreactor and the cell retention device.

2. The system of claim 1. wherein the first input reservoir contains fresh cell culture media.

3. The system of claim 1, wherein the second input reservoir contains transfection reagent mixture.

4. The system of claim 1, wherein the output reservoir collects spent cell growth medium and/or spent transfection reagent mixture.

5. The system of claim 1, wherein the cell retention device comprises at least one of a microfiltration cell retention device and an ultrafiltration cell retention device.

6. The system of claim 1, wherein the cell retention device operates in tangential flow filtration (TFF) mode.

7. The system of claim 1, wherein the cell retention device operates in alternating tangential flow (ATF) mode.

8. The system of claim 1, wherein each of the first pump, the second pump, and the third pump comprises a diaphragm pump, a centrifugal pump, a membrane pump, a peristaltic pump, and/or a positive displacement pump.

9. The system of claim 1, wherein each of the first valve, the second valve, and the third valve comprises a disposable valve.

10. The system of claim 1, wherein the continuous flow bioreactor has a capacity of about 0.015 L to about 2,000 L.

11. The system of claim 1, wherein the cell culture medium comprises a growth medium configured to culture eukaryotic cells comprising at least one of HEK-293 cells, Hela cells, Vero cells, BHK21 cells, and SF9 cells.

12. A method of producing viral vectors via cell transfection with addition of transfection reagent mixture at once, comprising:
(a) providing a cell culture system, comprising:
a continuous flow bioreactor:
a first input reservoir fluidly connected to the continuous flow bioreactor;
a cell retention device fluidly connected to the continuous flow bioreactor, the cell retention device comprising a retentate line and a permeate port;
a second input reservoir fluidly connected to the retentate line and to the continuous flow bioreactor; and
a spent media vessel fluidly connected to the cell retention device;
(b) performing cell culture of eukaryotic cells until a viable cell density (VCD) in the continuous flow bioreactor reaches a target value suitable for transfection;
(c) performing transfection of the cells; and
(d) producing virus in the continuous flow bioreactor by operating the bioreactor under virus production conditions.

13. The method of claim 12,
wherein performing transfection of the cells comprises continuously feeding transfection reagent mixture contained in the second input reservoir from the retentate line of the cell retention device and to the continuous flow bioreactor, and
wherein if perfusion after transfection is needed, the method further comprises continuously feeding a fresh supply of growth media through the continuous flow bioreactor while the spent media is continuously collected from the permeate of the cell retention device.

14. The method of claim 13, wherein the cell retention device is one of a microfiltration device or an ultrafiltration cell retention device.

15. The method of claim 13, wherein the cell retention device is operated in tangential flow filtration (TFF) mode.

16. The method of claim 13, wherein the cell retention device is operated in alternating tangential flow (ATF) mode.

17. The method of claim 13, wherein the method further comprises the steps of:
(e) if virus is excreted extracellularly, continuously harvesting excreted virus from the spent media collection vessel; and
(f) if virus is produced intracellularly, conducting a cell lysis procedure to release intracellular virus and conducting a clarification procedure by filtering cell lysate to remove cell debris and/or insoluble microparticles.

18. The method of claim 13, wherein the bioreactor has a capacity of about 0.015 L to about 2,000 L.

19. The method of claim 13, wherein the target VCD has a value of about 1,000,000 cells/mL to about 30,000,000 cells/mL.

20. The method of claim 13, wherein the cells comprise a member selected from the group consisting of HEK-293 cells, Hela cells, Vero cells. BHK21 cells, and SF9 cells.

21. The method of claim 13, wherein the virus comprise adeno-associated viruses (AAV), lentiviruses (LV), retroviruses (RV), adenoviruses (AV), and/or herpes simplex viruses (HSV).

22. A method of producing viral vectors via cell transfection with reduction of cell culture volume, comprising:
(a) providing a cell culture system, comprising:
a continuous flow bioreactor;
a cell retention device comprising a filtrate port that is fluidly connected to the continuous flow bioreactor; and
a spent media collection vessel fluidly connected to the cell retention device:
(b) performing cell culture of eukaryotic cells;
(c) circulating the cells into the cell retention device and returning the cells into the continuous flow bioreactor until a viable cell density (VCD) in the continuous flow bioreactor reaches a target value suitable for transfection;
(d) concentrating the cells, comprising:
starting operation of cell retention device to circulate the cells into the cell retention device and returning the cells into the continuous flow bioreactor while continuously collecting the spent media from the permeate of the cell retention device until the volume in the continuous flow bioreactor is reduced by an amount equal to a volume of transfection reagent mixture to be added;
(e) performing transfection of the cells, comprising the steps of:
(i) feeding the volume of transfection reagent mixture into the continuous flow bioreactor while the spent media is not collected from the permeate of the cell retention device; and
(ii) circulating the cells mixed with the transfection reagent mixture into the cell retention device and returning the cells into the continuous flow bioreactor for a period of time; and
(f) producing virus in the continuous flow bioreactor by operating the bioreactor under virus production conditions; and
(g) if perfusion after transfection is needed, continuously feeding a fresh supply of growth media through the continuous flow bioreactor while the spent media is continuously collected from the permeate of the cell retention device.

23. The method of claim 22, wherein the cell retention device is a microfiltration cell retention device.

24. The method of claim 22, wherein the cell retention device is an ultrafiltration cell retention device.

25. The method of claim 22, wherein the cell retention device is operated in tangential flow filtration (TFF) mode.

26. The method of claim 22, wherein the cell retention device is operated in alternating tangential flow (ATF) mode.

27. The method of claim 22, wherein the method further comprises the steps of:
(h) if virus is excreted extracellularly, continuously harvesting excreted virus from the spent media collection vessel; and
(i) if virus is produced intracellularly, conducting cell lysis to release intracellular virus and conducting clarification by filtering cell lysate to remove cell debris and/or insoluble microparticles.

28. The method of claim 22, wherein the cell culture system further comprises:
an input reservoir fluidly connected to the continuous flow bioreactor via an input valve and/or a feed pump,
wherein the input reservoir may hold fresh growth media or transfection reagent mixture.

29. £ The method of claim 22, wherein the bioreactor has a capacity of about 0.015 L to about 2,000 L.

30. £ The method of claim 22, wherein the target VCD has a value of about 1,000,000 cells/mL to about 30,000,000 cells/mL.

31. The method of claim 22, wherein the cells comprise a member selected from the group consisting of HEK-293 cells, Hela cells, Vero cells, BHK21 cells and SF9 cells.

32. The method of claim 22, wherein the virus comprises adeno-associated viruses (AAV), lentiviruses (LV), retroviruses (RV), adenoviruses (AV), and/or herpes simplex viruses (HSV).

33. The method of claim 22, wherein the period of time comprises at least 30 minutes.

34. A method of producing viral vectors via cell transfection with continuous addition of transfection reagent mixture at constant volume, comprising:
(a) providing a cell culture system, comprising:
a continuous flow bioreactor;
a cell retention device comprising a filtrate port that is fluidly connected to the continuous flow bioreactor; and
a spent media collection vessel fluidly connected to the cell retention device;
(b) performing cell culture of eukaryotic cells until a viable cell density (VCD) in the bioreactor reaches a target value suitable for transfection;
(c) performing transfection of the cells, comprising continuously feeding transfection reagent mixture at constant bioreactor volume using the feed pump until the transfection reagent mixture is completely transferred to the bioreactor;
(d) producing virus in the continuous flow bioreactor by operating the bioreactor under virus production conditions; and
(e) if perfusion after transfection is needed, continuously feeding a fresh supply of growth media through the continuous flow bioreactor while the spent media is continuously collected from the permeate of the cell retention device.

35. The method of claim 34, wherein the cell retention device is an ultrafiltration cell retention device.

36. The method of claim 34, wherein the cell retention device is operated in tangential flow filtration (TFF) mode.

37. The method of claim 34, wherein the cell retention device is operated in alternating tangential flow (ATF) mode.

38. The method of claim 34, wherein the method further comprises the steps of:

(l) if virus is excreted extracellularly, continuously harvesting excreted virus from the spent media collection vessel; and
(g) if virus is produced intracellularly, conducting a cell lysis procedure to release intracellular virus and conducting a clarification procedure by filtering cell lysate to remove cell debris and/or insoluble microparticles.

39. The method of claim 34, wherein the perfusion cell culture system further comprises:
an input reservoir fluidly connected to the continuous flow bioreactor via an input valve and/or a feed pump,
wherein the input reservoir may hold fresh growth media or transfection reagent mixture.

40. The method of claim 34, wherein the bioreactor has a capacity of about 0.015 L to about 2,000 L.

41. The method of claim 34, wherein the target VCD has a value of about 1,000,000 cells/mL to about 30,000,000 cells/mL.

42. The method of claim 34, wherein the cells comprise a member selected from the group consisting of HEK-293 cells, Hela cells, Vero cells, BHK21 cells and SF9 cells.

43. The method of claim 34, wherein the virus comprises adeno-associated viruses (AAV), lentiviruses (LV), retroviruses (RV), adenoviruses (AV), and/or herpes simplex viruses (HSV).

44. A method of producing viral vectors via cell transfection with continuous addition of transfection reagent mixture at constant bioreactor volume and continuous harvest of excreted virus during production phase, comprising:
(a) providing a cell culture system, comprising:
a continuous flow bioreactor;
a microfiltration cell retention device fluidly connected to the continuous flow bioreactor;
an ultrafiltration device fluidly connected to both the microfiltration cell retention device and the continuous flow bioreactor, the ultrafiltration device having a feed port, a retentate port, and a permeate port: and
a spent media collection vessel fluidly connected to both the microfiltration cell retention device and the ultrafiltration device;
(b) performing cell culture of eukaryotic cells until a viable cell density (VCD) in the continuous flow bioreactor reaches a target value suitable for transfection;
(c) performing transfection of the cells, comprising the steps of:
(i) continuously feeding transfection reagent mixture to the continuous flow bioreactor at a constant bioreactor volume for a period of time while continuously feeding the feed port of the ultrafiltration device from the permeate port of the microfiltration cell retention device;
(ii) at least partially permeating complexes of DNA and transfection reagents through the microfiltration cell retention device and collecting the complexes at the retentate port of the ultrafiltration device;
(iii) redirecting the complexes to the bioreactor; and
(iv) repeating steps (i)-(iii) until all the transfection reagent mixture is completely transferred to the continuous flow bioreactor; and
(d) producing virus in the continuous flow bioreactor by operating the bioreactor under virus production conditions; and
(e) if perfusion after transfection is needed, continuously feeding a fresh supply of growth media through the continuous flow bioreactor while the spent media is continuously collected from the permeate of the microfiltration cell retention device.

45. The method of claim 44, wherein the microfiltration cell retention device is operated in tangential flow filtration (TFF) mode.

46. The method of claim 44, wherein the microfiltration cell retention device is operated in alternating tangential flow (ATF) mode.

47. The method of claim wherein the method further comprises the steps of.
(f) if virus is excreted extracellularly, continuously harvesting excreted virus from the spent media collection vessel; and
(g) if virus is produced intracellularly, conducting a cell lysis procedure to release intracellular virus and conducting a clarification procedure by filtering cell lysate to remove cell debris and/or insoluble microparticles.

48. The method of claim 44, wherein the perfusion cell culture system further comprises:
an input reservoir fluidly connected to the continuous flow bioreactor via an input valve and/or a feed pump,
wherein the input reservoir may hold fresh growth media or transfection reagent mixture.

49. The method of claim 44, wherein the bioreactor has a capacity of about 0.015 L to about 2,000 L.

50. The method of claim 44, wherein the target VCD has a value of about 1,000,000 cells/mL to about 30,000,000 cells/mL.

51. The method of claim 44, wherein the cells comprise a member selected from the group consisting of HEK-293 cells, Hela cells, Vero cells, BHK21 cells, and SF9 cells.

52. The method of claim 44, wherein the virus comprise adeno-associated viruses (AAV), lentiviruses (LV), retroviruses (RV), adenoviruses (AV), and/or herpes simplex viruses (HSV).

53. A method of performing cell culture and cell transfection to produce viral vectors, comprising:
providing a cell culture system comprising a bioreactor with an input vessel;
providing eukaryotic cells to the bioreactor;
expanding the cells within the bioreactor for a period of time such that the growth of the cells is in a log phase until the viable cell density (VCD) reaches a target value;
initiating transfection on the cells by disposing a volume of transfection mix within the bioreactor in contact with the cells; and
performing at least one viral vector production step following transfection.

54. The method of claim 53, wherein initiating transfection comprises continuously adding transfection mix to the bioreactor where about 5% to about 95% of the total volume of the bioreactor is filled with cell culture media, and the volume of transfection mix added is about 5% to about 10 times the total volume of the bioreactor.

55. The method of claim 53, wherein the target VCD is from about 1,000,000 cells/mL to about 30,000,000 cells/mL.

56. The method of claim 53, comprising providing an ultrafiltration device fluidly coupled downstream of the bioreactor,
wherein gradually adding transfection mix comprises adding transfection to the bioreactor mix at a rate approximately equal to a filtrate flow rate of the ultrafiltration device. and
wherein gradually adding transfection mix comprises adding transfection mix over a period of from about 1 minute to about 30 minutes.

57. The method of claim 53, comprising concentrating the cells prior to initiating transfection, such that all of the viable cells of the cell culture are contained within a volumetric range from about 5% to about 75% of an interior volume of the bioreactor.

58. The method of claim 53, comprising providing an ultrafiltration device fluidly coupled downstream of the bioreactor,
wherein gradually adding transfection mix comprises adding transfection mix to the ultrafiltration cell at a rate faster than a filtrate flow rate of the ultrafiltration device, and wherein gradually adding transfection mix comprises adding transfection mix over a period of from about 1 minute to about 30 minutes.

59. The method of claim 53, comprising providing at least one of an ultrafiltration device and a microfiltration device fluidly coupled downstream of the bioreactor, the ultrafiltration device comprising at least one retentate line and at least one permeate line.

60. The method of claim 53, wherein initiating transfection comprises adding transfection mix to the retentate line from a second input vessel.

61. The method of claim 53, wherein the at least one viral vector production step comprises operating the bioreactor to culture cells that produce viruses, cell lysis, and/or clarification.

62. A cell culture system for producing viral vectors via cell transfection, comprising:
a continuous flow bioreactor;
an input reservoir fluidly connected to the continuous flow bioreactor via a first valve and/or a first pump;
a cell retention device fluidly connected to the continuous flow bioreactor via a second valve and/or a second pump; and
an output reservoir fluidly connected to the cell retention device via a third valve and/or a third pump.

63. The cell culture system of claim 62, wherein the cell retention device comprises at least one of a microfiltration cell retention device and an ultrafiltration cell retention device.

64. The cell culture system of claim 62, wherein the input reservoir contains fresh cell culture media and/or fresh transfection reagent mixture.

65. The cell culture system of claim 62, wherein the output reservoir collects spent cell culture media or spent transfection reagent mixture.

66. The cell culture system of claim 62, wherein the cell retention device is operating in tangential flow filtration (TFF) mode.

67. The cell culture system of claim 62, wherein the cell retention device is operating in alternating tangential flow (ATF) mode.

68. The cell culture system of claim 62, wherein each of the first pump, the second pump, and the third pump comprises a diaphragm pump, a centrifugal pump, a membrane pump, a peristaltic pump, or a positive displacement pump.

69. The cell culture system of claim 62, wherein each of the first valve, the second valve, and the third valve comprises a disposable valve.

70. A cell culture system for producing viral vectors via cell transfection, comprising:
a continuous flow bioreactor;
an input reservoir fluidly connected to the continuous flow bioreactor via a first valve and/or a first pump;
a microfiltration cell retention device fluidly connected to the continuous flow bioreactor via a second valve and/or a second pump:
an ultrafiltration device fluidly connected to both the microfiltration cell retention device and the continuous flow bioreactor via a third valve and/or a third pump; and
an output reservoir fluidly connected to both the ultrafiltration device and the third valve.

71. The cell culture system of claim 70, wherein the input reservoir contains fresh media or transfection mix.

72. The cell culture system of claim 70, wherein the output reservoir receives spent media.

73. The cell culture system of claim 70, wherein the system is operating in tangential flow filtration (TFF) mode.

74. The cell culture system of claim 70, wherein the system is operating in alternating tangential flow (ATF) mode.

75. The cell culture system of claim 70, wherein each of the first pump, the second pump, and the third pump comprises a diaphragm pump, a centrifugal pump, a membrane pump, a peristaltic pump, or a positive displacement pump.

76. The cell culture system of claim 70, wherein each of the first valve, the second valve, and the third valve comprises a disposable valve.

77. A cell culture system for producing viral vectors via cell transfection, comprising:
a continuous flow bioreactor;
a first input reservoir fluidly connected to the continuous flow bioreactor via a first valve and/or a first pump;
a cell retention device fluidly connected to the continuous flow bioreactor via a second valve and/or a second pump, the cell retention device comprising a retentate line;
a second input reservoir fluidly connected to the retentate line and fluidly connected to the continuous flow bioreactor; and
an output reservoir fluidly connected to the cell retention device via a third valve and/or a third pump.

78. The system of claim 77, wherein the cell retention device comprises at least one of a microfiltration cell retention device and an ultrafiltration cell retention device.

79. The system of claim 77, wherein the first input reservoir contains fresh cell culture media.

80. The system of claim 77, wherein the second input reservoir contains fresh transfection reagent mixture.

81. The system of claim 77, wherein the output reservoir collects spent cell culture media and/or spent transfection reagent mixture.

82. The system of claim 77, wherein each of the first pump, the second pump, and the third pump comprises a diaphragm pump, a centrifugal pump, a membrane pump, a peristaltic pump, or a positive displacement pump.

83. The system of claim 77, wherein each of the first valve, the second valve, and the third valve comprises a disposable valve.

84. The system of claim 77, wherein the cell retention device operates in tangential flow filtration (TFF) mode.

85. The system of claim 77, wherein the cell retention device operates in alternating tangential flow (ATF) mode.

86. A cell culture system for producing viral vectors via cell transfection, comprising:
a continuous flow bioreactor;
a first input reservoir fluidly connected to the continuous flow bioreactor via a first valve and/or first pump;
a cell retention device fluidly connected to the continuous flow bioreactor via a second valve and/or a second pump;
an output reservoir fluidly connected to the cell retention device via a third valve and/or third pump, the output reservoir for receiving permeate from the cell retention device;
a permeate outflow line for removing permeate from the cell retention device, the permeate outflow line fluidly coupling the cell retention device to the output reservoir; and
a second input reservoir fluidly coupled to both the continuous flow bioreactor and the permeate outflow line.

87. The system of claim 86, wherein during a transfection phase, permeate flows from the cell retention device to the output reservoir, and
wherein when the system is not in transfection phase, permeate flows from the cell retention device to the continuous flow bioreactor via the output reservoir.

88. The system of claim 86, comprising a three-way valve disposed within the permeate outflow line between the third pump and the output reservoir wherein, when the system is in transfection phase, the three-way valve diverts permeate to the output reservoir, and
wherein when the system is not in transfection phase, the three-way valve diverts permeate from the cell retention device to the continuous flow bioreactor via the second input reservoir.

89. The system of claim 86, wherein the transfection reagent mixture reservoir is fluidly coupled to the permeate outflow line downstream of the third pump and upstream of the spent media reservoir.

90. The system of claim 86, wherein the first input reservoir contains fresh cell culture media.

91. The system of claim 86, wherein the second input reservoir contains transfection reagent mixture.

92. The system of claim 86, wherein the output reservoir contains spent media.

93. A cell culture system for producing viral vectors via cell transfection, comprising:
a continuous flow bioreactor;
a cell retention device fluidly connected to the continuous flow bioreactor via a first valve and/or a first pump;
an output reservoir fluidly connected to the cell retention device via a second valve and/or a second pump, the spent media reservoir for receiving permeate from the cell retention device, and the second pump comprising double pump heads, a first flow stream and a second flow stream;
an input reservoir for containing at least one of transfection reagent mixture and fresh media, the input reservoir fluidly coupled to the continuous flow bioreactor via the first flow stream; and
a permeate outflow line for removing permeate from the cell retention device, the permeate outflow line fluidly coupling the cell retention device to the spent media reservoir via the second flow stream.

94. The system of claim 93, wherein the second pump comprises at least a first pump head and a second pump head,
wherein the first pump head pumps the first flow stream, and
wherein the second pump head pumps the second flow stream.

95. The system of claim 93, wherein the second pump comprises at least one of a diaphragm pump, a centrifugal pump, a membrane pump, a peristaltic pump, and a positive displacement pump.

96. The system of claim 93, wherein the first flow stream is fluidly isolated from the second flow stream.

97. The system of claim 93, wherein the first flow stream fluidly connects the input reservoir to the continuous flow bioreactor, and wherein the second flow stream fluidly connects the permeate of the cell retention device to the output reservoir.

98. The system of claim 93, wherein the output reservoir collects spent cell culture media and/or spent transfection reagent mixture.
